(19) United States
(12) Patent Application Publication
(10) Pub. No.: US 2019/0040408 A1
(43) Pub. Date: Feb. 7, 2019

(54) MUTATED HYDROXYPHENYLPYRUVATE DIOXYGENASE, DNA SEQUENCE AND ISOLATION OF PLANTS WHICH ARE TOLERANT TO HPPD INHIBITOR HERBICIDES

(71) Applicants: BAYER CROPSCIENCE NV, Diegem (BE); BAYER INTELLECTUAL PROPERTY GMBH, Monheim Am Rhein (DE)

(72) Inventors: Marco BUSCH, Schwalbach Am Taunus (DE); Kerstin SELAK, Alzey-Weinheim (DE); Bernd LABER, Idstein (DE); Alain SAILLAND, Saint Didier Au Mont D'or (FR)

(21) Appl. No.: 15/895,573

(22) Filed: Feb. 13, 2018

Related U.S. Application Data
(63) Continuation of application No. 14/256,798, filed on Apr. 18, 2014, now abandoned.
Application No. 14/256,798 is a continuation of application No. 12/937,812, filed on Oct. 14, 2010, now abandoned.
(60) Provisional application No. 61/124,082, filed on Apr. 14, 2008.

(30) Foreign Application Priority Data
Apr. 14, 2008 (EP) 08154481.9

(51) Int. Cl. C12N 15/82; C12N 9/02

(52) U.S. Cl. CPC C12N 15/8274; C12Y 113/11027; C12N 9/0069

(57) ABSTRACT

The present invention relates to a nucleic acid sequence encoding a mutated hydroxyphenylpyruvate dioxygenase (HPPD), to a chimeric gene which comprises this sequence as the coding sequence, and to its use for obtaining plants which are resistant to HPPD inhibitor herbicides.

### RELATED APPLICATIONS

This application is a continuation application of application Ser. No. 12/937,812 filed Oct. 14, 2010, which is a national stage application (under 35 U.S.C. § 371) of PCT/EP2009/054343, filed Apr. 10, 2009, which claims priority of European application 08154481.9 filed Apr. 14, 2008, and U.S. Provisional application 61/124,082, filed Apr. 14, 2008. The entire contents of each of these applications are hereby incorporated by reference herein in their entirety.

### SUBMISSION OF SEQUENCE LISTING

The Sequence Listing associated with this application is filed in electronic format via EFS-Web and hereby incorporated by reference into the specification in its entirety. The name of the text file containing the Sequence Listing is 5500_ 187_Sequence_Listing. The size of the text file is 88 KB, and the text file was created on Apr. 18, 2014.

The present invention relates to a nucleic acid sequence encoding a mutated hydroxyphenylpyruvate dioxygenase (HPPD), to a chimeric gene which comprises this sequence as the coding sequence, and to its use for obtaining plants which are resistant to HPPD inhibitor herbicides.

The hydroxyphenylpyruvate dioxygenases (HPPD; EC 1.13.11.27) are enzymes which catalyse the reaction in which para-hydroxyphenylpyruvate (HPP), a tyrosine degradation product, is transformed into homogentisate (HG), the precursor in plants of tocopherol and plastoquinone (Crouch N. P. et al., 1997; Fritze et al., 2004). Tocopherol acts as a membrane-associated antioxidant. Plastoquinone, firstly acts as an electron carrier between PSII and the cytochrome b6/f complex and secondly, is a redox cofactor for phytoene desaturase, which is involved in the biosynthesis of carotenoids.

Most plants synthesize tyrosine via arrogenate (Abou-Zeid et al. 1995; Bonner et al., 1995; Byng et al., 1981; Connely and Conn 1986; Gaines et al., 1982). In these plants, the HPP is derived only from the degradation of tyrosine. On the other hand, in organisms such as the yeast Sacharomyces cerevisiae or the bacterium Escherichia coli, HPP is a tyrosine precursor, and it is synthesized by the action of an enzyme, prephenate dehydrogenase (hereinafter referred to as PDH), which converts prephenate to HPP (Lingens et al., 1967; Sampathkumar and Morrisson 1982). In these organisms, the production of HPP is therefore directly connected to the aromatic amino acid biosynthetic pathway (shikimate pathway), and not to the tyrosine degradation pathway.

Inhibition of HPPD leads to uncoupling of photosynthesis, deficiency in accessory light-harvesting pigments and, most importantly, to destruction of chlorophyll by UV-radiation and reactive oxygen species due to the lack of photo protection normally provided by carotenoids (Norris et al. 1995). Photo bleaching of photosynthetically active tissues leads to growth inhibition and plant death.

Some molecules which inhibit HPPD, and which bind specifically to the enzyme in order to inhibit transformation of the HPP into homogentisate, have proven to be very effective selective herbicides.

Most commercially available HPPD inhibitor herbicides belong to one of these four chemical families:

1) the triketones, e.g. sulcotrione [i.e. 2-[2-chloro-4-(methylsulfonyl)benzoyl]-1,3-cyclohexanedione], mesotrione [i.e. 2-[4-(methylsulfonyl)-2-nitrobenzoyl]-1,3-cyclohexanedione], tembotrione [i.e. 2-[2-chloro-4-(methylsulfonyl)-3-[(2,2,2,-tri-fluoroethoxy)methyl]benzoyl]-1,3-cyclo-hexanedione];
2) The diketonitriles, e.g. 2-cyano-3-cyclopropyl-1-(2-methylsulphonyl-4-trifluoromethylphenyl)-propane-1,3-dione and 2-cyano-1-[4-(methylsulphonyl)-2-trifluoromethylphenyl]-3-(1-methylcyclopropyl)propane-1,3-fione;
2) the isoxazoles, e.g. isoxaflutole [i.e. (5-cyclopropyl-4-isoxazolyl)[2-(methylsulfonyl)-4-(trifluoromethyl)phenyl]methanone]. In plants, the isoxaflutole is rapidly metabolized in DKN, a diketonitrile compound which exhibits the HPPD inhibitor property; and
4) the pyrazolinates, e.g. topramezone [i.e. [3-(4,5-dihydro-3-isoxazolyl)-2-methyl-4-(methylsulfonyl) phenyl] (5-hydroxy-1-methyl-1H-pyrazol-4-yl)methanone], and pyrasulfotole [(5-hydroxy-1,3-dimethylpyrazol-4-yl(2-mesyl-4-trifluaromethylphenyl)methanone].

These HPPD-inhibiting herbicides can be used against grass and/or broad leaf weeds in crop plants that display metabolic tolerance, such as maize (Zea mays) in which they are rapidly degraded (Schulz et al., 1993; Mitchell et al., 2001; Garcia et al., 2000; Pallett et al., 2001). In order to extend the scope of these HPPD-inhibiting herbicides, several efforts have been developed in order to confer to plants, particularly plants without or with an underperforming metabolic tolerance, an agricultural level tolerance to them.

Besides the attempt of by-passing HPPD-mediated production of homogentisate (U.S. Pat. No. 6,812,010), overexpressing the sensitive enzyme so as to produce quantities of the target enzyme in the plant which are sufficient in relation to the herbicide has been performed (WO96/38567). Overexpression of HPPD resulted in better pre-emergence tolerance to the diketonitrile derivative (DKN) of Isoxaflutole (IFT), but tolerance was not sufficient for tolerance to post-emergence treatment (Matringe et al., 2005).

A third strategy was to mutate the HPPD in order to obtain a target enzyme which, while retaining its properties of catalysing the transformation of HPP into homogentisate, is less sensitive to HPPD inhibitors than is the native HPPD before mutation. This strategy has been successfully applied for the production of plants tolerant to 2-cyano-3-cyclopropyl-1-(2-methylsulphonyl-4-trifluoromethylphenyl)-propane-1,3-dione and to 2-cyano-1-[4-(methylsulphonyl)-2-trifluoromethylphenyl]-3-(1-methylcyclopropyl)propane-1,3-fione (EP496630), two HPPD-inhibiting herbicides belonging to the diketonitriles family (WO 99/24585). Pro215Leu, Gly336Glu, Gly336Ile, and more particularly Gly336Trp (positions of the mutated amino acid are indicated with reference to the Pseudomonas HPPD of SEQ ID NO:2) were identified as mutations which are responsible for an increased tolerance to pre-emergence treatment with these diketonitrile herbicides without causing an alteration of the activity of the enzyme.

More recently, introduction of a Pseudomonas HPPD gene into the plastid genome of tobacco and soybean has shown to be much more effective than nuclear transformation, conferring even tolerance to post-emergence application of isoxaflutol (Dufourmantel et al., 2007).

In WO 04/024928, the inventors have sought to increase the prenylquinone biosynthesis (e.g., synthesis of plastoquinones, tocopherols) in the cells of plants by increasing the flux of the HPP precursor into the cells of these plants. This has been done by connecting the synthesis of said precursor to the “shikimate” pathway by overexpression of a PDH enzyme. They have also noted that the transformation of plants with a gene encoding a PDH enzyme makes it possible to increase the tolerance of said plants to HPPD inhibitors.

Despite these successes obtained for the development of plants showing tolerance to diketonitrile herbicides, it is still necessary to develop and/or improve the system of tolerance to HPPD inhibitors, particularly for HPPD inhibitors belonging to the classes of the triketones (e.g. sulcotrione, mesotrione, and tembotrione) and the pyrazolinates (e.g. topramezone and pyrasulfotole).

The present invention therefore relates to novel mutated HPPD enzymes which retain their properties of catalysing the conversion of para-hydroxyphenylpyruvate (HPP) to homogentisate and which are less sensitive to HPPD inhibitors than the original unmutated HPPD, characterized in that they contain a mutation at the position 336 (amino acid glycine in the native HPPD) with reference to the Pseudomonas HPPD of SEQ ID NO:2 which is selected from the following mutations: Gly336Arg, Gly336His, Gly336Met, Gly336Phe, Gly336Asn, Gly336Cys, Gly336Val, Gly336Trp, Gly336Glu and Gly336Asp.

In a particular embodiment, the mutation in the position 336 with reference to the Pseudomonas HPPD of SEQ ID NO:2 is selected from the following mutations: Gly336Arg, Gly336His, Gly336Met, Gly336Phe, Gly336Asn, Gly336Cys, and Gly336Val, provided that the mutated HPPD is not the double mutant Gly334Ala-Gly336Arg (positions are given with reference to the Pseudomonas HPPD of SEQ ID NO:2).

In a more particular embodiment, the mutation in the position 336 with reference to the Pseudomonas HPPD of SEQ ID NO:2 is selected from the following mutations: Gly336His, Gly336Met, Gly336Cys, and Gly336Phe.

In another particular embodiment, the HPPD enzyme is from a plant, particularly from Arabidopsis thaliana, and contains a mutation on glycine at position 422 with reference to the amino acid sequence of the Arabidopsis HPPD of SEQ ID NO:4 (i.e. position 336 with reference to the amino acid sequence of the Pseudomonas HPPD of SEQ ID NO:2) which is selected from the following mutations: Gly336Arg, Gly336His, Gly336Met, Gly336Phe, Gly336Asn, Gly336Cys, Gly336Val, Gly336Trp, Gly336Glu and Gly336Asp.

In a more particular embodiment, the mutation in position 422 with reference to the Arabidopsis HPPD of SEQ ID NO:4 (i.e. in the position 336 with reference to the Pseudomonas HPPD of SEQ ID NO:2) is selected from the following mutations: Gly336His, Gly336Asn, Gly336Cys, and Gly336Val, and the mutated HPPD is of plant origin, particularly from Arabidopsis. It is noted than the position 336 with reference to the Pseudomonas HPPD of SEQ ID NO:2 is the position 422 with reference to the Arabidopsis thaliana HPPD of SEQ ID NO:4.

In a particular embodiment, the mutated HPPD of the invention is less sensitive than the original unmutated HPPD to a HPPD inhibitor herbicide of the class of isoxazoles, diketonitriles, triketones or pyrazolinates.

In a particular embodiment, the mutated HPPD of the invention is less sensitive than the original unmutated HPPD to a HPPD inhibitor herbicide selected from isoxaflutole, tembotrione, mesotrione, sulcotrione, pyrasulfotole, Topramezone, 2-cyano-3-cyclopropyl-1-(2-SO₂CH₃-4-CF₃phenyl)propane-1,3-dione and 2-cyano-3-cyclopropyl-1-(2-SO₂CH₃-4-2,3 Cl₂ phenyl)propane-1,3-dione.

In another particular embodiment, the mutated HPPD of the invention is less sensitive to an HPPD inhibitor of the class of triketones (named triketone HPPD inhibitor), such as tembotrione, sulcotrione and mesotrione, particularly tembotrione, or of the class of pyrazolinates (named pyrazolinate HPPD inhibitor), such as pyrasulfotole and topramezone, than the original unmutated HPPD.

In a more particular embodiment, the mutated HPPD of the invention is less sensitive to a triketone HPPD inhibitor selected from tembotrione, sulcotrione and mesotrione, particularly tembotrione.

In another particular embodiment, the mutated HPPD of the invention contains a second mutation, in addition to the first mutation on the amino acid glycine at the position 336 with reference to the Pseudomonas HPPD of SEQ ID NO:2.

In a more particular embodiment, the second mutated amino acid is selected from the selected amino acids: Pro215, Gly298, Gly332, Phe333, Gly334 and Asn337, with reference to the Pseudomonas HPPD sequence of SEQ ID NO:2.

Also, the present invention provides mutated HPPD enzymes which retain their properties of catalysing the conversion of para-hydroxyphenylpyruvate (HPP) to homogentisate and which are less sensitive to HPPD inhibitors of the class of triketones such as tembotrione, sulcotrione and mesotrione, or of the class of pyrazolinates such as pyrasulfotole and topramezone, than the original unmutated HPPD, characterized in that they contain a mutation of the amino acid glycine at the position 336 with reference to the Pseudomonas HPPD of SEQ ID NO:2, as well as uses of such enzymes to render plants tolerant to these HPPD inhibitors, processes wherein triketones or pyrazolinates herbicides are applied to plants expressing such mutant enzymes, and plants tolerant to such HPPD inhibitors of the class of triketones or pyrazolinates by comprising in their genome a gene encoding certain HPPD enzymes mutated in the position 336 with reference to the Pseudomonas HPPD of SEQ ID NO:2.

In a particular embodiment of the invention, the mutated HPPD enzyme is less sensitive to a HPPD inhibitor of the class of triketones such as tembotrione, sulcotrione and mesotrione than the original unmutated HPPD and is mutated in the position 336 with reference to the Pseudomonas HPPD of SEQ ID NO:2 according to a mutation selected from the following mutations: Gly336Arg, Gly336Asp, Gly336Glu, Gly336His, Gly336Met, Gly336Phe, Gly336Trp, Gly336Asn, Gly336Cys and Gly336Val.

In a particular embodiment of the invention, the mutated HPPD enzyme is less sensitive to a HPPD inhibitor of the class of triketones such as tembotrione, sulcotrione and mesotrione than the original unmutated HPPD and is mutated in the position 336 with reference to the Pseudomonas HPPD of SEQ ID NO:2 according to a mutation selected from the following mutations: Gly336His, Gly336Met, Gly336Phe, and Gly336Cys.

Several HPPDs and their primary sequences have been described in the state of the art, in particular the HPPDs of bacteria such as Pseudomonas (Rüetschi et al., Eur. J. Biochem., 205, 459-466, 1992, WO 96/38567), of plants such as Arabidopsis (WO 96/38567, Genebank AF047834), carrot (WO 96/38567, Genebank 87257), Avena sativa (WO 02/046387), wheat (WO 02/046387), Brachiaria platyphylla (WO 02/046387), Cenchrus echinatus (WO 02/046387), Lolium rigidum (WO 02/046387), Festuca arundinacea (WO 02/046387), Setaria faberi (WO 02/046387), Eleusine indica (WO 02/046387), and Sorghum (WO 02/046387), of Coccicoides (Genebank COITRP) or of mammals such as the mouse or the pig. The corresponding sequences disclosed in the indicated references are hereby incorporated by reference.

By aligning these known sequences, by using the customary means of the art, such as, for example, the method described by Thompson, J. D. et al. (CLUSTAL W: improving the sensitivity of progressive multiple sequence alignment through sequence weighting, positions-specific gap penalties and weight matrix choice. Nucleic Acids Research, 22; 4673-4680, 1994), and accessing these computer programs for sequence alignment which are accessible via the Internet, for example, the skilled person is able to define the sequence homologies in relation to a reference sequence and find the key amino acids or else define common regions.

In the case of the present invention, the reference sequence is the Pseudomonas sequence, with all the definitions and indications of the positions of particular amino acids being made with respect to the primary Pseudomonas HPPD sequence of SEQ ID NO: 2, except when specifically indicated. The attached FIG. 1 depicts an alignment of several HPPD sequences which are described in the state of the art; these sequences are aligned with respect to the Pseudomonas HPPD sequence as the reference sequence and comprise the HPPD sequences of Streptomyces avermitilis (Genebank SAV11864), of Daucus carota (Genebank DCU 87257), of Arabidopsis thaliana (Genebank AF047834), of Zea mais, of Hordeum vulgare (Genebank HVAJ693), of Mycosphaerella graminicola (Genebank AF038152), of Coccicoides immitis (Genebank COITRP) and of Mus musculus (Genebank MU54HD) This figure gives the numbering of the amino acids of the Pseudomonas sequence and also the amino acids which are common to these sequences, with these amino acids being designated by an asterisk. On the basis of such an alignment, it is easy, from the definition of the Pseudomonas amino acid by its position and its nature, to identify the position of the corresponding amino acid in another HPPD sequence. FIG. 1 shows that this can be done with the alignment of sequences of different plant, mammalian and bacterial origin, demonstrating that this method of alignment, which is well known to a skilled person, can be generalized to any other sequence. An alignment of different HPPD sequences is also described in Patent Application WO 97/49816.

In WO99/24585, the analysis of the tertiary structure of the Pseudomonas HPPD monomer shows the presence of a C-terminal part of the HPPDs, which is where the active site of the enzyme is located, linked to its N-terminal part by a linking peptide which ensures the stability of the enzyme and its oligomerization (the Pseudomonas HPPD is a tetramer while the plant HPPDs are dimers). This structure was obtained by the customary methods of studying crystal X-ray diffraction. The linking peptide makes it possible to define the N-terminal end of the C-terminal part of the enzyme, with the said linking peptide being located between amino acids 145 and 157 in the case of Pseudomonas (cf. FIG. 1). Two amino acids, which are in positions 161 and 162 in the case of the Pseudomonas sequence (D=Asp161 and H=His162), will be noted in all sequences shown in the sequence alignment depicted in the attached FIG. 1. With reference to the Pseudomonas HPPD, it is therefore possible to define the linking peptide as being located between approximately 5 and 15 amino acids upstream of the amino acid Asp161.

According to the invention, “mutated HPPD” is understood as being the replacement of at least one amino acid of the primary sequence of the HPPD with another amino acid. The expression “mutated amino acid” will be used below to designate the amino acid which is replaced by another amino acid, thereby designating the site of the mutation in the primary sequence of the protein.

According to the invention, the mutation is effected on the amino acid glycine at position 336 with reference to the Pseudomonas sequence of SEQ ID NO: 2, which is common to almost all the identified HPPD sequences. On 240 HPPD sequences known so far, 238 contain a glycine at position 336, and only the HPPD sequences of Synechococcus sp. JA-3-3Ab (Acc-No Q2JX04) and Synechococcus sp. JA-2-3B′a(2-13) (Acc-No Q2JPN8)) have an alanine at this postion. Gly336 is part of a consensus sequence “Gly-Phe-Gly-X-Gly-Asn-Phe” found in most of the HPPD sequences, wherein X can be any of the 20 amino acids, among the HPPDs from various origins, which makes the identification of the Gly336 feasible without any difficulties in HPPDs from any source by the sequence alignment method.

As an example, Gly336 with reference to the Pseudomonas sequence is Gly422 with reference to the Arabidopsis thaliana sequence of SEQ ID NO: 4 (see FIG. 1), but herein reference will be made to Gly at reference position 336 by reference to the Pseudomonas sequence of SEQ ID NO: 2 (except when specifically indicated), even though the mutation can be in any useful HPPD enzyme in accordance with this invention, not necessarily in the Pseudomonas HPPD.

The enzymatic activity of HPPDs can be measured by any method that makes it possible either to measure the decrease in the amount of the HPP or O₂ substrates, or to measure the accumulation of any of the products derived from the enzymatic reaction, i.e. homogentisate or CO₂. In particular, the HPPD activity can be measured by means of the method described in Garcia et al. (1997) or Garcia et al. (1999), which are incorporated herein by reference.

According to the invention, a HPPD inhibitor of the class of triketones (or triketone HPPD inhibitor) means a HPPD inhibitor having a triketone skeleton. As an example of such triketone HPPD inhibitor, one can cite the molecules sulcotrione [i.e. 2-[2-chloro-4-(methylsulfonyl)benzoyl]-1,3-cyclohexanedione], mesotrione [i.e. 2-[4-(methylsulfonyl)-2-nitrobenzoyl]-1,3-cyclohexanedione], and tembotrione [i.e. 2-[2-chloro-4-(methylsulfonyl)-3-[(2,2,2,-tri-fluoroethoxy)methyl]benzoyl]-1,3-cyclo-hexanedione].

According to the invention, a HPPD of the class of pyrazolinates (or pyrazolinate HPPD inhibitor) means a HPPD inhibitor having a pyrazole radical. As an example of such pyrazolinates HPPD inhibitor, one can cite the molecules topramezone [i.e. [3-(4,5-dihydro-3-isoxazolyl)-2-methyl-4-(methylsulfonyl)phenyl](5-hydroxy-1-methyl-1H-pyrazol-4-yl)methanone] and pyrasulfotole [(5-hydroxy-1,3-dimethylpyrazol-4-yl(2-mesyl-4-trifluaromethylphenyl)methanone].

In a further embodiment of the invention, HPPD is mutated at a second amino acid position in addition to the mutation of Gly336. The presence of this second mutation may further increase the tolerance to the same HPPD inhibitor herbicide than the one for which the first mutation is conferring a tolerance, or may confer tolerance to a second HPPD inhibitor herbicide. Examples of such mutations conferring tolerance to HPPD inhibitors, and in particular to diketonitriles and to the isoxaflutole, are described in WO 99/24585.

In a particular embodiment of the invention, the second mutated amino acid is selected from the following reference amino acids, with reference to the Pseudomonas sequence of SEQ ID NO: 2: Pro215, Gly332, Phe333, Gly334 and Asn337, and also Gly298 in the Pseudomonas sequence (this last having no counterpart in other HPPDs, see FIG. 1).

In one embodiment of the invention, the second mutated amino acid is Pro215 with reference to the Pseudomonas sequence of SEQ ID NO: 2, and the mutation is particularly Pro215Leu.

The present invention also relates to a nucleic acid sequence, particularly an isolated DNA, which encodes a mutated HPPD as described above.

The present invention also relates to a nucleic acid sequence encoding a mutated HPPD enzyme which retains their properties of catalysing the conversion of para-hydroxyphenylpyruvate (HPP) to homogentisate and which is less sensitive to HPPD inhibitors of the class of triketones such as tembotrione, sulcotrione and mesotrione, or of the class of pyrazolinates such as pyrasulfotole and topramezone, than the original unmutated HPPD, characterized in that it contains a mutation of the amino acid glycine at the position 336 with reference to the Pseudomonas HPPD of SEQ ID NO:2.

In a more particular embodiment, the nucleic acid sequence of the invention encodes a mutated HPPD enzyme which is less sensitive to a HPPD inhibitor of the class of triketones such as tembotrione, sulcotrione and mesotrione than the original unmutated HPPD and wherein the HPPD is mutated in the position 336 with reference to the Pseudomonas HPPD of SEQ ID NO:2 according to a mutation selected from the following mutations: Gly336Arg, Gly336Asp, Gly336Glu, Gly336His, Gly336Met, Gly336Phe, Gly336Trp, Gly336Asn, Gly336Cys and Gly336Val.

In an even more particular embodiment, the nucleic acid sequence of the invention encodes a mutated HPPD enzyme which is less sensitive to a HPPD inhibitor of the class of triketones such as tembotrione, sulcotrione and mesotrione than the original unmutated HPPD and wherein the HPPD is mutated in the position 336 with reference to the Pseudomonas HPPD of SEQ ID NO:2 according to a mutation selected from the following mutations: Gly336His, Gly336Met, Gly336Phe, and Gly336Cys.

According to the present invention, a “nucleic acid sequence” is understood as being a nucleotide sequence which can be of the DNA or RNA type, preferably of the DNA type, and in particular double-stranded, whether it be of natural or synthetic origin, in particular a DNA sequence in which the codons which encode the mutated HPPD according to the invention have been optimized in accordance with the host organism in which it is to be expressed (e.g., by replacing codons with those codons more preferred or most preferred in codon usage tables of such host organism or the group to which such host organism belongs, compared to the original host), with these methods of optimization being well known to the skilled person.

An “isolated DNA”, as used herein, refers to a DNA which is not naturally-occurring or no longer in the natural environment wherein it was originally present, e.g., a DNA coding sequence associated with other regulatory elements in a chimeric gene, a DNA transferred into another host cell, such as a plant cell, or an artificial, synthetic DNA having a different nucleotide sequence compared to any known naturally-occurring DNA.”

The sequence which encodes an original unmutated HPPD which will be mutated according to the invention, can be of any origin whatever. In particular, it can be of bacterial origin. Advantageous examples which may be cited are bacteria of the Pseudomonas sp. type, for example Pseudomonas fluorescens, or otherwise cyanobacteria of the Synechocystis genus. The sequence can also be of plant origin, in particular derived from dicotyledonous plants, umbelliferous plants, or otherwise monocotyledonous plants. Advantageous examples which may be cited are plants such as tobacco, Arabidopsis, Daucus carotta, Zea mais (corn), wheat, barley, Avena sativa, wheat, Brachiaria platyphylla, Cenchrus echinatus, Lolium rigidum, Festuca arundinacea, Setaria faberi, Eleusine indica, and Sorghum. The coding sequences, and the way of isolating and cloning them, are described in the previously cited references, the contents of which are hereby incorporated by reference.

In a particular embodiment of the invention, the HPPD is from a bacterial origin, particularly from Pseudomonas sp., more particularly from Pseudomonas fluorescens, or from a plant origin, particularly from Arabidopsis thaliana.

The HPPD to make the mutation(s) in for the purpose of the invention, can be any naturally-occurring HPPD, or any active fragment thereof or any variant thereof wherein some amino acids (1 to 10 amino acids) have been replaced, added or deleted for cloning purposes, to make a transit peptide fusion, and the like, which retains HPPD activity, i.e. the property of catalysing the conversion of para-hydroxyphenylpyruvate to homogentisate.

According to the invention, the HPPD may be a chimeric HPPD. The term “chimeric HPPD” is intended to mean an HPPD comprising elements originating from various HPPDs. Such chimeric HPPDs are in particular described in patent application WO 99/24586.

The mutation can be effected in the nucleic acid sequence which encodes the original unmutated HPPD by any means which is appropriate for replacing, in the said sequence, the codon which encodes the mutated amino acid with the codon which corresponds to the amino acid which is to replace it, with the said codons being widely described in the literature and well known to the skilled person.

Several molecular biological methods can be used to achieve this mutation.

A preferred method for preparing a mutated nucleic acid sequence according to the invention, and the corresponding protein, comprises carrying out site-directed mutagenesis on codons encoding one or more amino acids which are selected in advance, including the codon for reference position Gly336 with reference to the Pseudomonas HPPD sequence of SEQ ID NO:2. The methods for obtaining these site-directed mutations are well known to the skilled person and widely described in the literature (in particular: Directed Mutagenesis: A Practical Approach, 1991, Edited by M. J. McPHERSON, IRL PRESS), or are methods for which it is possible to employ commercial kits (for example the U.S.E. mutagenesis kit from PHARMACIA). After the site-directed mutagenesis, it is useful to select the cells which contain a mutated HPPD which is less sensitive to an HPPD inhibitor by using an appropriate screening aid. One screening method which is simple to implement is to determine the dose of HPPD inhibitor which fully inhibits the original unmutated HPPD, and which is lethal for the cells which express this unmutated HPPD, and to subject the mutated cells to this predetermined dose, and thereafter to isolate the mutated cells which have withstood this lethal dose, and then to isolate and to clone the gene which encodes the mutated HPPD. In view of a particular embodiment of the invention and the sought-after solution, i.e. an HPPD which is less sensitive to a triketone or pyrazolinate HPPD inhibitor, the screening may be performed as described above using a triketone or a pyrazolinate HPPD inhibitor, particularly an HPPD inhibitor selected from tembotrione, mesotrione, pyrasulfotole, topramezone and sulcotrione.

In view of another embodiment of the invention, i.e. an HPPD which is further mutated on a second amino acid, in addition to the first mutation on the reference amino acid in position 336 with reference to the Pseudomonas HPPD sequence of SEQ ID NO:2, the second mutation may be obtained by site-directed mutagenesis, performed simultaneously or successively to the first one.

As an alternative to the site-directed mutagenesis as described above, the second mutation may be obtained using methods of random mutation (such as EMS or radiation treatment) associated with an appropriate screening aid. Such methods of mutation are well known to the skilled person, and are amply described in the literature (in particular: Sambrook et al., 1989). Screening methods can be performed as described above.

The terminology DNA or protein “comprising” a certain sequence X, as used throughout the text, refers to a DNA or protein including or containing at least the sequence X, so that other nucleotide or amino acid sequences can be included at the 5′ (or N-terminal) and/or 3′ (or C-terminal) end, e.g. (the nucleotide sequence of) a selectable marker protein, (the nucleotide sequence of) a transit peptide, and/or a 5′ leader sequence or a 3′ trailer sequence. Similarly, use of the term “comprise”, “comprising” or “comprises” throughout the text and the claims of this application should be understood to imply the inclusion of a stated integer or step or group of integers or steps but not the exclusion of any other integer or step or group of integers or steps

The present invention therefore also relates to a method for preparing a nucleic acid sequence which encodes a mutated HPPD according to the invention, with the said method being defined above.

The invention also relates to the use, in a method for transforming plants, of a nucleic acid which encodes a mutated HPPD according to the invention as a marker gene or as a coding sequence which makes it possible to confer to the plant tolerance to herbicides which are HPPD inhibitors, and the use of HPPD inhibitors on plants comprising a nucleic acid sequence encoding a mutated HPPD according to the invention. In an embodiment of this invention, in such use the HPPD inhibitors are triketones or pyrazolinates, preferably tembotrione, mesotrione or sulcotrione. It is of course understood that this sequence can also be used in combination with (an)other gene marker(s) and/or sequence(s) which encode(s) one or more protein with useful agricultural properties.

Among the genes which encode proteins that confer useful agronomic properties on the transformed plants, mention can be made of the DNA sequences encoding proteins which confer tolerance to certain herbicides, those which confer tolerance to certain insects, those which confer tolerance to certains diseases, etc. . . . . Such genes are in particular described in Patent Applications WO 91/02071 and WO95/06128. Among the DNA sequences encoding proteins which confer tolerance to certain herbicides on the transformed plant cells and plants, mention can be made of the bar gene which confers tolerance to glufosinate herbicides, the gene encoding a suitable EPSPS which confers tolerance to herbicides having EPSPS as a target, such as glyphosate and its salts (U.S. Pat. No. 4,535,060, U.S. Pat. No. 4,769,061, U.S. Pat. No. 5,094,945, U.S. Pat. No. 4,940,835, U.S. Pat. No. 5,188,642, U.S. Pat. No. 4,971,908, U.S. Pat. No. 5,145,783, U.S. Pat. No. 5,310,667, U.S. Pat. No. 5,312,910, U.S. Pat. No. 5,627,061, U.S. Pat. No. 5,633,435), the gene encoding glyphosate oxydoreductase (U.S. Pat. No. 5,463,175).

Among the DNA sequences encoding a suitable EPSPS which confer tolerance to the herbicides which have EPSPS as a target, mention will more particularly be made of the gene which encodes a plant EPSPS, in particular maize EPSPS, which has two mutations, 102 and 106, and which is described in Patent Application FR 2 736 926, hereinafter named EPSPS double mutant, or the gene which encodes an EPSPS isolated from agrobacterium and which is described by sequence ID No. 2 and sequence ID No. 3 of U.S. Pat. No. 5,633,435, hereinafter named CP4.

In the cases of the DNA sequences encoding EPSPS, and more particularly encoding the genes above, the sequence encoding these enzymes is advantageously preceded by a sequence encoding a transit peptide, in particular encoding the “optimized transit peptide” described in U.S. Pat. No. 5,510,471 or 5,633,448.

Among the DNA sequences encoding proteins of interest which confer novel properties of tolerance to insects, mention will more particularly be made of the Bt proteins widely described in the literature and well known to those skilled in the art. Mention will also be made of proteins extracted from bacteria such as Photorhabdus (WO 97/17432 & WO 98/08932).

The present invention also relates to a chimeric gene (or expression cassette) which comprises a coding sequence as well as heterologous regulatory elements, at the 5′ and/or 3′ position, at least at the 5′ position, which are able to function in a host organism, in particular plant cells or plants, with the coding sequence containing at least one nucleic acid sequence which encodes a mutated HPPD as previously defined.

The present invention therefore relates to a chimeric gene (or expression cassette) which comprises a coding sequence as well as heterologous regulatory elements, at the 5′ and/or 3′ position, at least at the 5′ position, which are able to function in a host organism, in particular plant cells or plants, with the coding sequence containing at least one nucleic acid sequence as previously defined.

In a particular embodiment, the present invention relates to a chimeric gene as previously described, wherein the host organism is selected from bacteria, yeasts, Pichia, fungi, baculovirus, plant cells and plants.

In another particular embodiment, the present invention relates to a chimeric gene as previously described, wherein the chimeric gene contains in the 5′ position of the nucleic acid sequence which encodes a mutated HPPD, a nucleic acid sequence which encodes a plant transit peptide, with this sequence being arranged between the promoter region and the sequence encoding the mutated HPPD so as to permit expression of a transit peptide/mutated HPPD fusion protein.

As a regulatory sequence which is a promoter in plant cells and plants, use may be made of any promoter sequence of a gene which is naturally expressed in plants, in particular a promoter which is expressed especially in the leaves of plants, such as for example “constitutive” promoters of bacterial, viral or plant origin, or “light-dependent” promoters, such as that of a plant ribulose-biscarboxylase/oxygenase (RuBisCO) small subunit gene, or any suitable known promoter which may be used. Among the promoters of plant origin, mention will be made of the histone promoters as described in Application EP 0 507 698, or the rice actin promoter (U.S. Pat. No. 5,641,876). Among the promoters of a plant virus gene, mention will be made of that of the cauliflower mosaic virus (CAMV 19S or 35S), or the circovirus promoter (AU 689 311).

Use may also be made of a regulatory promoter sequence specific for particular regions or tissues of plants, such as promoters specific for seeds (Datla, R. et al., 1997), especially the napin promoter (EP 255 378), the phaseolin promoter, the glutenin promoter, the helianthinin promoter (WO 92/17580), the albumin promoter (WO 98/45460), the oleosin promoter (WO 98/45461), the SAT1 promoter or the SAT3 promoter (PCT/US98/06978).

Use may also be made of an inducible promoter advantageously chosen from the phenylalanine ammonia lyase (PAL), HMG-CoA reductase (HMG), chitinase, glucanase, proteinase inhibitor (PI), PR1 family gene, nopaline synthase (nos) and vspB promoters (U.S. Pat. No. 5,670,349, Table 3), the HMG2 promoter (U.S. Pat. No. 5,670,349), the apple beta-galactosidase (ABG1) promoter and the apple aminocyclopropane carboxylate synthase (ACC synthase) promoter (WO 98/45445).

According to the invention, use may also be made, in combination with the promoter, of other regulatory sequences, which are located between the promoter and the coding sequence, such as transcription activators (“enhancers”), for instance the translation activator of the tobacco mosaic virus (TMV) described in Application WO 87/07644, or of the tobacco etch virus (TEV) described by Carrington & Freed 1990, for example, or introns such as the adh1 intron of maize or intron 1 of rice actin.

As a regulatory terminator or polyadenylation sequence, use may be made of any corresponding sequence of bacterial origin, such as for example the nos terminator of Agrobacterium tumefaciens, of viral origin, such as for example the CaMV 35S terminator, or of plant origin, such as for example a histone terminator as described in Application EP 0 633 317.

“Host organism” is understood as being any unicellular or multicellular organism into which the chimeric gene according to the invention can be introduced for the purpose of producing mutated HPPD. These organisms are, in particular, bacteria, for example E. coli, yeasts, in particular of the genera Saccharomyces or Kluyveromyces, Pichia, fungi, in particular Aspergillus, a baculovirus or, preferably, plant cells and plants.

“Plant cell” is understood, according to the invention, as being any cell which is derived from or found in a plant and which is able to form or is part of undifferentiated tissues, such as calli, differentiated tissues such as embryos, parts of plants, plants or seeds.

“Plant” is understood, according to the invention, as being any differentiated multicellular organism which is capable of photosynthesis, in particular a monocotyledonous or dicotyledonous organism, more especially cultivated plants which are or are not intended for animal or human nutrition, such as maize or corn, wheat, Brassica spp. plants such as Brassica napus or Brassica juncea, soybean, rice, sugarcane, beetroot, tobacco, cotton, vegetable plants such as cucumber, leek, carrot, tomato, lettuce, peppers, melon, watermelon, etc.

In one embodiment the invention relates to the transformation of plants. Any promoter sequence of a gene which is expressed naturally in plants, or any hybrid or combination of promoter elements of genes expressed naturally in plants, including Agrobacterium or plant virus promoters, or any promoter which is suitable for controlling the transcription of a herbicide tolerance gene, can be used as the promoter regulatory sequence in the plants of the invention. Examples of such suitable promoters are described above.

According to the invention, it is also possible to use, in combination with the promoter regulatory sequence, other regulatory sequences which are located between the promoter and the coding sequence, such as intron sequences, or transcription activators (enhancers). Examples of such suitable regulatory sequences are described above.

Any corresponding sequence of bacterial origin, such as the nos terminator from Agrobacterium tumefaciens, or of plant origin, such as a histone terminator as described in application EP 0 633 317, may be used as transcription termination (and polyadenylation) regulatory sequence.

In one particular embodiment of the invention, a nucleic acid sequence which encodes a transit peptide is employed 5′ of the nucleic acid sequence encoding a mutated HPPD, with this transit peptide sequence being arranged between the promoter region and the sequence encoding the mutated HPPD so as to permit expression of a transit peptide/mutated HPPD fusion protein, with the mutated HPPD being previously defined. The transit peptide makes it possible to direct the mutated HPPD into the plastids, more especially the chloroplasts, with the fusion protein being cleaved between the transit peptide and the mutated HPPD when the latter enters the plastid. The transit peptide may be a single peptide, such as an EPSPS transit peptide (described in U.S. Pat. No. 5,188,642) or a transit peptide of that of the plant ribulose biscarboxylase/oxygenase small subunit (RuBisCO ssu), where appropriate including a few amino acids of the N-terminal part of the mature RuBisCO ssu (EP 189 707), or else may be a fusion of several transit peptides such as a transit peptide which comprises a first plant transit peptide which is fused to a part of the N-terminal sequence of a mature protein having a plastid location, with this part in turn being fused to a second plant transit peptide as described in patent EP 508 909, and, more especially, the optimized transit peptide which comprises a transit peptide of the sunflower RuBisCO ssu fused to 22 amino acids of the N-terminal end of the maize RuBisCO ssu, in turn fused to the transit peptide of the maize RuBisCO ssu, as described, with its coding sequence, in patent EP 508 909.

The present invention also relates to the transit peptide/mutated HPPD fusion protein and a nucleic acid or plant-expressible chimeric gene encoding such fusion protein, wherein the two elements of this fusion protein are as defined above.

The present invention also relates to a cloning and/or expression vector for transforming a host organism, which vector contains at least one chimeric gene as defined above. In addition to the above chimeric gene, this vector contains at least one origin of replication. This vector can be a plasmid, a cosmid, a bacteriophage or a virus which has been transformed by introducing the chimeric gene according to the invention. Such transformation vectors, which depend on the host organism to be transformed, are well known to the skilled person and widely described in the literature. The transformation vector which is used, in particular, for transforming plant cells or plants may be a virus, which can be employed for transforming developed plants and which additionally contains its own replication and expression elements. According to the invention, the vector for transforming plant cells or plants is preferably a plasmid, such as a disarmed Agrobacterium Ti plasmid.

The present invention also relates to the host organisms, in particular plant cells or plants, which are transformed and which contain a chimeric gene which comprises a sequence encoding a mutated HPPD as defined above, and the use of the plants of the invention in a field to grow a crop and harvest a plant product, e.g., soybean or corn grains, where in one embodiment said use involves the application of HPPD inhibitor herbicides to such plants to control weeds. In one embodiment of this invention, in such use the HPPD inhibitors are triketones or pyrazolinates, preferably tembotrione, mesotrione or sulcotrione, particularly tembotrione.

Therefore, the present invention relates to a host organism, in particular a plant cell or plant, characterized in that it contains at least one chimeric gene as previously described above, or at least a nucleic acid sequence as previously described.

In a particular embodiment, the present invention relates to a plant cell or plant characterized in that it contains at least a nucleic acid sequence which encodes a mutated HPPD enzyme which retain its properties of catalysing the conversion of para-hydroxyphenylpyruvate (HPP) to homogentisate and which is less sensitive to an HPPD inhibitor than the original unmutated HPPD, characterized in that it contains a mutation at the position 336 (amino acid glycine in the native HPPD) with reference to the Pseudomonas HPPD of SEQ ID NO:2 which is selected from the following mutations: Gly336Arg, Gly336His, Gly336Met, Gly336Phe, Gly336Asn, Gly336Cys, and Gly336Val, provided that the mutated HPPD is not the double mutant Gly334Ala-Gly336Arg (positions are given with reference to the Pseudomonas HPPD of SEQ ID NO:2).

In a further more particular embodiment, the present invention relates to a plant cell or plant characterized in that it contains at least a nucleic acid sequence which encodes a mutated HPPD as described above, wherein the mutation in the position 336 with reference to the Pseudomonas HPPD of SEQ ID NO:2 is selected from the following mutations: Gly336His, Gly336Met, Gly336Cys, and Gly336Phe, particularly Gly336His.

In another particular embodiment, the present invention relates to a plant cell or plant characterized in that it contains at least a nucleic acid sequence which encodes a mutated HPPD which retain its properties of catalysing the conversion of para-hydroxyphenylpyruvate (HPP) to homogentisate and which is less sensitive to an HPPD inhibitor than the original unmutated HPPD, wherein the HPPD enzyme is from a plant, particularly from Arabidopsis thaliana, and contains a mutation on glycine at position 422 with reference to the amino acid sequence of the Arabidopsis HPPD of SEQ ID NO:4 (i.e. position 336 with reference to the amino acid sequence of the Pseudomonas HPPD of SEQ ID NO:2) selected from the following mutations: Gly336Arg, Gly336His, Gly336Met, Gly336Phe, Gly336Asn, Gly336Cys, Gly336Val, Gly336Trp, Gly336Glu and Gly336Asp.

In a further more particular embodiment, the present invention relates to a plant cell or plant characterized in that it contains at least a nucleic acid sequence which encodes a mutated HPPD as described above, wherein the mutation in the position 336 with reference to the Pseudomonas HPPD of SEQ ID NO:2 is selected from the following mutations: Gly336His, Gly336Asn, Gly336Cys, and Gly336Val, and the mutated HPPD is of plant origin, particularly from Arabidopsis. It is noted than the position 336 with reference to the Pseudomonas HPPD of SEQ ID NO:2 is the position 422 with reference to the Arabidopsis thaliana HPPD of SEQ ID NO:4

In a particular embodiment, the present invention relates to a plant cell or plant characterized in that it contains at least a nucleic acid sequence which encodes a mutated HPPD as described above, wherein the mutated HPPD of the invention is less sensitive than the original unmutated HPPD to a HPPD inhibitor herbicide of the class of isoxazoles, diketonitriles, triketones or pyrazolinates.

In a more particular embodiment, the present invention relates to a plant cell or plant characterized in that it contains at least a nucleic acid sequence which encodes a mutated HPPD as described above, wherein the mutated HPPD is less sensitive than the original unmutated HPPD to a HPPD inhibitor herbicide selected from isoxaflutole, tembotrione, mesotrione, sulcotrione, pyrasulfotole, Topramezone, 2-cyano-3-cyclopropyl-1-(2-SO₂CH₃-4-CF₃phenyl)propane-1,3-dione and 2-cyano-3-cyclopropyl-1-(2-SO₂CH₃-4-2,3 Cl₂ phenyl)propane-1,3-dione.

In another particular embodiment, the present invention relates to a plant cell or plant characterized in that it contains at least a nucleic acid sequence which encodes a mutated HPPD as described above, wherein the mutated HPPD is less sensitive to an HPPD inhibitor of the class of triketones such as tembotrione, sulcotrione and mesotrione, particularly tembotrione, or of the class of pyrazolinates such as pyrasulfotole and topramezone, than the original unmutated HPPD.

In a more particular embodiment, the present invention relates to a plant cell or plant characterized in that it contains at least a nucleic acid sequence which encodes a mutated HPPD as described above, wherein the mutated HPPD is less sensitive to a triketone HPPD inhibitor selected from tembotrione, sulcotrione and mesotrione, particularly tembotrione.

In another particular embodiment, the present invention relates to a plant cell or plant characterized in that it contains at least a nucleic acid sequence which encodes a mutated HPPD as described above, wherein the mutated HPPD of the invention contains a second mutation, in addition to the first mutation on the amino acid glycine at the position 336 with reference to the Pseudomonas HPPD of SEQ ID NO:2.

In a more particular embodiment, the present invention relates to a plant cell or plant characterized in that it contains at least a nucleic acid sequence which encodes a mutated HPPD as described above, wherein the second mutated amino acid is selected from the selected amino acids: Pro215, Gly298, Gly332, Phe333, Gly334 and Asn337, with reference to the Pseudomonas HPPD sequence of SEQ ID NO:2.

The present invention further relates to a plant cell or plant characterized in that it contains at least a nucleic acid sequence which encodes a mutated HPPD enzyme which retains their properties of catalysing the conversion of para-hydroxyphenylpyruvate (HPP) to homogentisate and which is less sensitive to HPPD inhibitors of the class of triketones such as tembotrione, sulcotrione and mesotrione, or of the class of pyrazolinates such as pyrasulfotole and topramezone, than the original unmutated HPPD, characterized in that it contains a mutation of the amino acid glycine at the position 336 with reference to the Pseudomonas HPPD of SEQ ID NO:2.

In a more particular embodiment, the present invention relates to a plant cell or plant characterized in that it contains at least a nucleic acid sequence which encodes a mutated HPPD enzyme which is less sensitive to a HPPD inhibitor of the class of triketones or pyrazolinates than the original unmutated HPPD is mutated in the position 336 with reference to the Pseudomonas HPPD of SEQ ID NO:2 according to a mutation selected from the following mutations: Gly336Arg, Gly336Asp, Gly336Glu, Gly336His, Gly336Met, Gly336Phe, Gly336Trp, Gly336Asn, Gly336Cys and Gly336Val.

In another particular embodiment, the present invention relates to a plant cell or plant characterized in that it contains at least a nucleic acid sequence as previously described, and in addition a gene that is functional in plants, allowing overexpression of a PDH (prephenate dehydrogenase) enzyme.

The present invention also relates to the plants which contain transformed cells, in particular the plants which are regenerated from the transformed cells. The regeneration can be obtained by any appropriate method, with the method depending on the nature of the species, as described, for example, in the above references. The following patents and patent applications may be cited, in particular, with regard to the methods for transforming plant cells and regenerating plants: U.S. Pat. No. 4,459,355, U.S. Pat. No. 4,536,475, U.S. Pat. No. 5,464,763, U.S. Pat. No. 5,177,010, U.S. Pat. No. 5,187,073, EP 267,159, EP 604 662, EP 672 752, U.S. Pat. No. 4,945,050, U.S. Pat. No. 5,036,006, U.S. Pat. No. 5,100,792, U.S. Pat. No. 5,371,014, U.S. Pat. No. 5,478,744, U.S. Pat. No. 5,179,022, U.S. Pat. No. 5,565,346, U.S. Pat. No. 5,484,956, U.S. Pat. No. 5,508,468, U.S. Pat. No. 5,538,877, U.S. Pat. No. 5,554,798, U.S. Pat. No. 5,489,520, U.S. Pat. No. 5,510,318, U.S. Pat. No. 5,204,253, U.S. Pat. No. 5,405,765, EP 442 174, EP 486 233, EP 486 234, EP 539 563, EP 674 725, WO 91/02071 and WO 95/06128.

The present invention also relates to the transformed plants or part thereof, which are derived by cultivating and/or crossing the above regenerated plants, and to the seeds of the transformed plants.

The present invention also relates to the end products such as the meal or oil which are obtained from the plants, part thereof, or seeds of the invention.

The transformed plants which can be obtained in accordance with the invention can be of the monocotyledonous type, such as cereals, sugarcane, rice and corn or maize, or of the dicotyledonous type, such as tobacco, soybean, Brassica spp. plants such as oilseed rape, cotton, beetroot, clover, etc.

The invention relates to a method for transforming host organisms, in particular plant cells or plants, by integrating in such organisms at least one nucleic acid sequence or one chimeric gene as previously defined, wherein it is possible to obtain the transformation by any appropriate known means, which means are amply described in the specialist literature and, in particular, the references cited in the present application, more especially by using the vector according to the invention.

One series of methods comprises bombarding cells, protoplasts or tissues with particles to which the DNA sequences are attached. Another series of methods comprises using, as the means for transfer into the plant, a chimeric gene which is inserted into an Agrobacterium tumefaciens Ti plasmid or an Agrobacterium rhizogenes Ri plasmid. Other methods may be used, such as microinjection or electroporation or otherwise direct precipitation using PEG. The skilled person can select any appropriate method for transforming the host organism of choice, in particular the plant cell or the plant. As examples, the technology for soybean transformation has been extensively described in the examples 1 to 3 of EP 1186666, incorporated herein by reference. For rice, agrobacterium-mediated transformation (Hiei et al., 1994, and Hiei et al., 1997, incorporated herein by reference), electroporation (U.S. Pat. No. 5,641,664 and U.S. Pat. No. 5,679,558, incorporated herein by reference), or bombardment (Christou et al., 1991, incorporated herein by reference) could be performed. A suitable technology for transformation of monocotyledonous plants, and particularly rice, is described in WO 92/09696, incorporated herein by reference. For cotton, agrobacterium-mediated transformation (Gould J. H. and Magallanes-Cedeno M., 1998 and Zapata C., 1999, incorporated herein by reference), polybrene and/or treatment-mediated transformation (Sawahel W. A., 2001, incorporated herein by reference) have been described.

In a particular embodiment of the invention, the mutated HPPD is targeted into the chloroplast. This may be done by integrating a nucleic acid sequence which encodes a transit peptide/mutated HPPD fusion protein as described above.

Alternatively, the mutated HPPD may be expressed directly in the chloroplasts using transformation of the chloroplast genome. A suitable method comprises the bombardment of leaf sections by particles coated with the DNA and integration of the introduced gene encoding the protein of the invention by homologous recombination. Suitable vectors and selection systems are known to the person skilled in the art. An example of means and methods which can be used for such integration into the chloroplast genome of tobacco lines is given in WO 06/108830, the content of which are hereby incorporated by reference. When the polypeptides are directly targeted to the chloroplast using transformation of the chloroplast genome, a transit peptide sequence is generally not required.

The present invention also relates to a method for obtaining a plant resistant to an HPPD inhibitor, characterized in that the plant is transformed with a chimeric gene as previously described.

Therefore, the present invention also relates to a method for obtaining a plant resistant to an HPPD inhibitor, characterized in that the plant is transformed with a chimeric gene which comprises a coding sequence as well as heterologous regulatory element in the 5′ and optionally in the 3′ positions, which are able to function in a host organism, characterized in that the coding sequence contains at least a nucleic acid sequence as previously described.

In a particular embodiment of this invention, in this method the HPPD inhibitor is a triketone or pyrazolinate herbicide, preferably tembotrione, mesotrione or sulcotrione, particularly tembotrione.

In another particular embodiment, the present invention relates to a method for obtaining a plant resistant to an HPPD inhibitor as described above, characterized in that the plant is further transformed, simultaneously or successively, with a gene functional in this plant allowing overexpression of a PDH (prephenate dehydrogenase) enzyme.

The invention also relates to a method for selectively weeding plants, in particular plant crops, with the aid of an HPPD inhibitor, in particular a herbicide as previously defined, which method is characterized in that this herbicide is applied to plants which have been transformed in accordance with the invention, either before sowing the crop, before emergence of the crop or after emergence of the crop.

In a particular embodiment of this invention, in this method the HPPD inhibitor is a triketone or pyrazolinate herbicide, preferably tembotrione, mesotrione or sulcotrione, particularly tembotrione.

The invention also relates to a method for controlling weeds in an area or a field which contains transformed seeds as previously described in the present patent application, which method comprises applying, to the said area of the field, a dose of a HPPD inhibitor herbicide which is toxic for the said weeds, without significantly affecting the seeds or plants which contains a nucleic acid sequence or a chimeric gene as previously described in the present patent application.

In a particular embodiment of this invention, in this method the HPPD inhibitor is a triketone or pyrazolinate herbicide, preferably tembotrione, mesotrione or sulcotrione, particularly tembotrione.

The present invention also relates to a method for cultivating the plants which have been transformed with a chimeric gene according to the invention, which method comprises planting seeds comprising a chimeric gene of the invention, in an area of a field which is appropriate for cultivating the said plants, and in applying, if weeds are present, a dose, which is toxic for the weeds, of a herbicide whose target is the above-defined HPPD to the said area of the said field, without significantly affecting the said transformed seeds or the said transformed plants, and in then harvesting the cultivated plants or plant parts when they reach the desired stage of maturity and, where appropriate, in separating the seeds from the harvested plants.

In a particular embodiment of this invention, in this method the HPPD inhibitor is a triketone or pyrazolinate herbicide, preferably tembotrione, mesotrione or sulcotrione, particularly tembotrione.

In the above methods, the herbicide whose target is the HPPD can be applied in accordance with the invention, either before sowing the crop, before the crop emerges or after the crop emerges.

The present invention also relates to a process for obtaining oil, particularly soybean oil, or meal, comprising growing a crop, particularly a soybean crop, expressing a mutated HPPD of the invention in a field, optionally treating such crop with an HPPD inhibitor herbicide, harvesting the grains and milling the grains to make meal and extract the oil. Also the plants seeds or grains, either whole, broken or crushed, containing the chimeric gene of the invention are part of this invention.

Therefore, the present invention relates to a method for obtaining oil or meal comprising growing a transformed plant as described above, optionally treating such plant with an HPPD inhibitor herbicide, harvesting the grains and milling the grains to make meal and extract the oil.

In particular embodiments, the above methods of the invention are involving an HPPD inhibitor herbicide selected from isoxaflutole, tembotrione, mesotrione, pyrasulfotole, sulcotrione, topramezone, 2-cyano-3-cyclopropyl-1-(2-SO₂CH₃-4-CF₃phenyl)propane-1,3-dione and 2-cyano-3-cyclopropyl-1-(2-SO₂CH₃-4-2,3 Cl₂ phenyl)propane-1,3-dione.

In other particular embodiments, the above methods of the invention are involving an HPPD inhibitor herbicide of the class of triketones, such as tembotrione, sulcotrione and mesotrione, or of the class of pyrazolinates, such as pyrasulfotole and topramezone, particularly selected from tembotrione, sulcotrione and mesotrione, more particularly tembotrione.

Within the meaning of the present invention, “herbicide” is understood as being a herbicidally active substance on its own or such a substance which is combined with an additive which alters its efficacy, such as, for example, an agent which increases its activity (a synergistic agent) or which limits its activity (a safener). It is of course to be understood that, for their application in practice, the above herbicides are combined, in a manner which is known per se, with the formulation adjuvants which are customarily employed in agricultural chemistry.

When the plant which has been transformed in accordance with the invention contains one or more other genes for tolerance towards other herbicides (as, for example, a gene which encodes a mutated or unmutated EPSPS which confers on the plant tolerance to glyphosate herbicides or a pat or bar gene conferring tolerance to glufosinate herbicides), or when the transformed plant is naturally sensitive to another herbicide (such as sulfonylurea tolerance), the method according to the invention can comprise the simultaneous or chronologically staggered application of an HPPD inhibitor in combination with the said herbicide or herbicide combination, for example glyphosate and/or glufosinate and/or sulfonylurea herbicides.

The invention also relates to the use of the chimeric gene encoding a mutated HPPD according to the invention as a marker gene during the transformation of a plant species, based on the selection on the abovementioned HPPD inhibitor herbicides.

The present invention also relates to a method for obtaining a plant resistant to a triketone or a pyrazolinate HPPD inhibitor, characterized in that the plant is transformed with a chimeric gene expressing in the plant a HPPD mutated in the amino acid glycine at position 336 with reference to the amino acid sequence of the Pseudomonas HPPD of SEQ ID NO: 2.

In a particular embodiment, the invention relates to said method for obtaining a plant resistant to a triketone or a pyrazolinate HPPD inhibitor, characterized in that the HPPD mutation is selected from Gly336Arg, Gly336Asp, Gly336Glu, Gly336His, Gly336Met, Gly336Phe, Gly336trp, Gly336Asn, Gly336Cys, and Gly336Val.

In another particular embodiment, the invention relates to said method for obtaining a plant resistant to a triketone HPPD inhibitor selected from tembotrione, mesotrione and sulcotrione.

In another particular embodiment, the invention relates to said method for obtaining a plant resistant to a triketone or a pyrazolinate HPPD inhibitor, characterized in that the plant is further transformed, simultaneously or successively, with a gene functional in this plant allowing overexpression of a PDH (prephenate dehydrogenase) enzyme.

The invention also relates to a method for controlling weeds in an area or a field, which method comprises planting in this area or field transformed plants resistant to a triketone or a pyrazolinate HPPD 3,5 inhibitor which has been obtained according to the method described above, or transformed seeds which originates from them, and in applying a dose which is toxic for the weeds of said triketone or pyrazolinate HPPD inhibitor without significantly affecting the said transformed seeds or the said transformed plants.

The invention also relates to a method for obtaining oil or meal comprising growing a transformed plant resistant to a triketone or a pyrazolinate HPPD inhibitor which has been obtained according to the method described above, or a transformed seed which originates from such plant, optionally treating such plant or seed with a triketone or a pyrazolinate HPPD inhibitor, harvesting the grains and milling the grains to make meal and extract the oil.

The invention also relates to the use of a HPPD which has been mutated in the amino acid glycine at the position 336 with reference to the amino acid sequence of the Pseudomonas HPPD of SEQ ID NO:2 to render plants tolerant to a triketone or a pyrazolinate HPPD inhibitor.

The invention also relates to the use of a mutated HPPD as described above, characterized in that the HPPD mutation is selected from Gly336Arg, Gly336Asp, Gly336Glu, Gly336His, Gly336Met, Gly336Phe, Gly336trp, Gly336Asn, Gly336Cys, Gly336Val.

The invention also relates to the use of a mutated HPPD as described above, characterized in that the HPPD inhibitor is a triketone HPPD inhibitor selected from tembotrione, mesotrione, and sulcotrione.

The present invention also relates to a host organism, in particular plant cells or plants, which contain a chimeric gene comprising a sequence encoding a mutated HPPD according to the invention, and which also contain a gene functional in this host organism allowing overexpression of a prephenate dehydrogenase (abbreviated herein as PDH) enzyme.

In the expression “gene that is functional in plants, allowing overexpression of a PDH enzyme”, the term “PDH” should be interpreted as referring to any natural or mutated PDH enzyme exhibiting the PDH activity of conversion of prephenate to HPP. In particular, said PDH enzyme can originate from any type of organism. An enzyme with PDH activity can be identified by any method that makes it possible either to measure the decrease in the amount of prephenate substrate, or to measure the accumulation of a product derived from the enzymatic reaction, i.e. HPP or one of the cofactors NADH or NADPH. In particular, the PDH activity can be measured by means of the method described in example 4.

Many genes encoding PDH enzymes are described in the literature, and their sequences can be identified on the website http://www.ncbi.nlm.nih.gov/entrez/. Particularly known is the gene encoding the PDH enzyme of the yeast Saccharomyces cerevisiae (Accession No. S46037) as described in Mannhaupt et al. (1989), of a bacterium of the Bacillus genus, in particular of the species B. subtilis (Accession No. P20692) as described in Henner et al. (1986), of a bacterium of the Escherichia genus, in particular of the species E. coli (Accession No. KMECTD) as described in Hudson et al. (1984), or of a bacterium of the Erwinia genus, in particular of the species E. herbicola (Accession No. S29934) as described in Xia et al. (1992).

The invention further relates to a method for obtaining a host organism, particularly a plant cell or a plant, resistant to an HPDD inhibitor by integrating in such organism at least one nucleic acid sequence or one chimeric gene as defined above, and by further transforming it, simultaneously or successively, with a gene functional in this host organism allowing overexpression of a PDH (prephenate dehydrogenase) enzyme.

In a particular embodiment, the invention relates to a method for obtaining a host organism, particularly a plant cell or a plant, resistant to a triketone or pyrazolinate HPDD inhibitor, particularly tembotrione, mesotrione or sulcotrione.

Means and methods which could be used for obtaining a host organisms, particularly a plant cell or a plant, transformed both with a gene allowing overexpression of an HPPD enzyme, and with a gene allowing overexpression of a PDH enzyme are extensively described in WO 04/024928, the content of which is hereby incorporated by reference.

The reference in this specification to any prior publication (or information derived from it), or to any matter which is known, is not, and should not be taken as an acknowledgement or admission or any form of suggestion that that prior publication (or information) or known matter forms part of the common general knowledge in the field of this invention.

### FIGURES

FIG. 1: Alignment the HPPD sequences of Streptomyces avermitilis, Daucus carota, Arabidopsis thaliana, Zea mais, Hordeum vulgare, Mycosphaerella graminicola, Coccicoides immitis, Mus musculus, and Pseudomonas fluorescens. The numbering of the amino acids is done according to the Pseudomonas sequence, and an asterisk designates the amino acids which are common to these sequences.

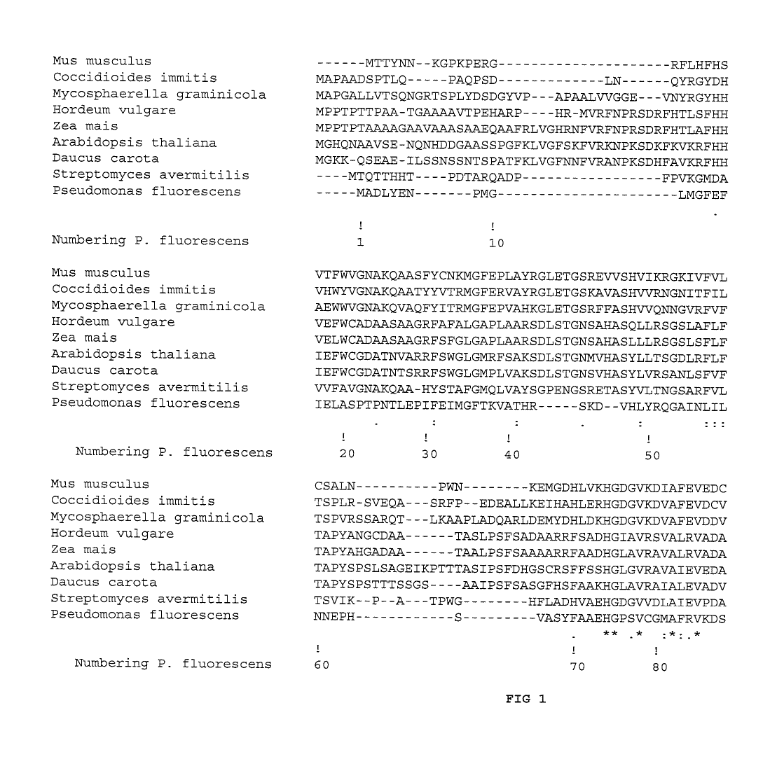

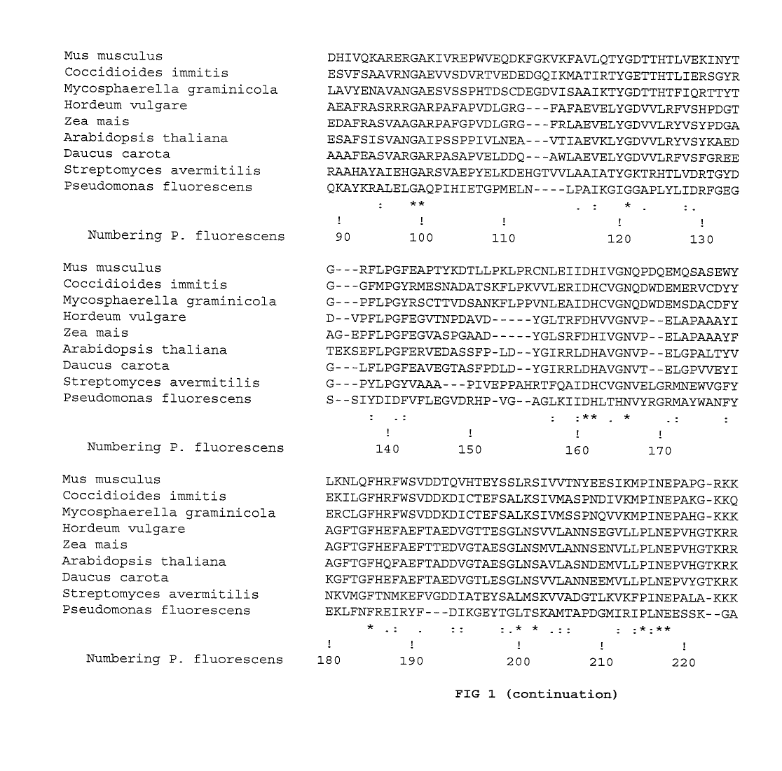

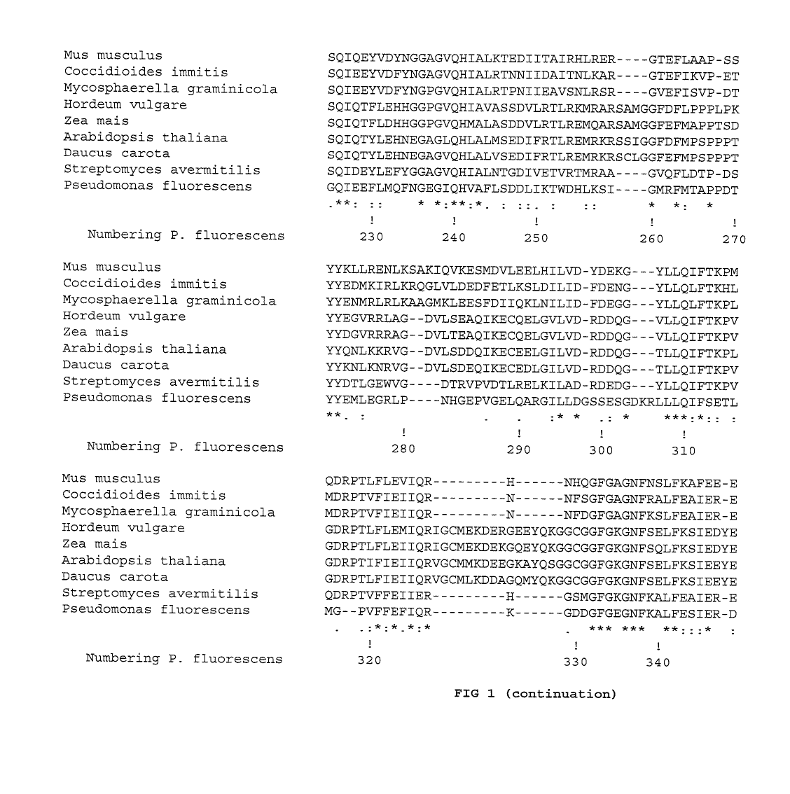

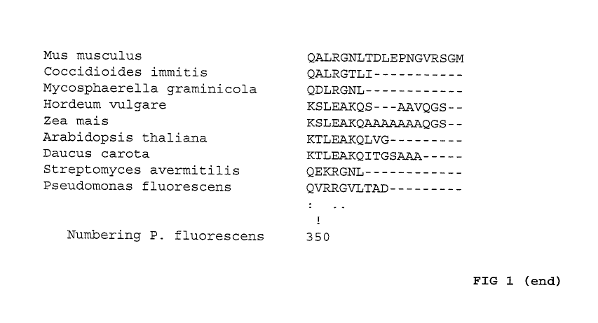

### SEQUENCES LISTING

SEQ ID NO 1: Nucleic acid sequence encoding Pseudomonas fluorescens HPPD

SEQ ID NO 2: Pseudomonas fluorescens HPPD amino acid sequence

SEQ ID NO 3: Nucleic acid sequence encoding Arabidopsis thaliana HPPD

SEQ ID NO 4: Arabidopsis thaliana HPPD amino acid sequence

SEQ ID NO 5: Nucleic acid sequence encoding Mus musculus HPPD

SEQ ID NO 6: Mus musculus HPPD amino acid sequence

SEQ ID NO 7: Nucleic acid sequence encoding Coccidioides immitis HPPD

SEQ ID NO 8: Coccidioides immitis HPPD amino acid sequence

SEQ ID NO 9: Nucleic acid sequence encoding Mycosphaerella graminicola HPPD

SEQ ID NO 10: Mycosphaerella graminicola HPPD amino acid sequence

SEQ ID NO 11: Nucleic acid sequence encoding Hordeum vulgare HPPD

SEQ ID NO 12: Hordeum vulgare HPPD amino acid sequence

SEQ ID NO 13: Nucleic acid sequence encoding Zea mais HPPD

SEQ ID NO 14: Zea mais HPPD amino acid sequence

SEQ ID NO 15: Nucleic acid sequence encoding Daucus carota HPPD

SEQ ID NO 16: Daucus carota HPPD amino acid sequence

SEQ ID NO 17: Nucleic acid sequence encoding Streptomyces avermitilis HPPD

SEQ ID NO 18: Streptomyces avermitilis HPPD amino acid sequence

SEQ ID NO 19: primer sequence kerfi001

SEQ ID NO 20: primer sequence kerfi002

SEQ ID NO 21: primer sequence kerfi003

SEQ ID NO 22: primer sequence kerfi004

SEQ ID NO 23: primer sequence kerfi007

SEQ ID NO 24: primer sequence kerfi008

SEQ ID NO 25: primer sequence kerfi011

SEQ ID NO 26: primer sequence kerfi012

SEQ ID NO 27: primer sequence kerfi014

SEQ ID NO 28: primer sequence kerfi016

SEQ ID NO 29: primer sequence kerfi019

SEQ ID NO 30: primer sequence kerfi020

SEQ ID NO 31: primer sequence kerfi015

SEQ ID NO 32: primer sequence kerfi018

### EXAMPLES

The various aspects of the invention will be better understood with the aid of the experimental examples which follow. All the methods or operations which are described below in these examples are given by way of example and correspond to a choice which is made from among the different methods which are available for arriving at the same or similar result. This choice has no effect on the quality of the result and, as a consequence, any suitable method can be used by the skilled person to arrive at the same or similar result. The majority of the methods for manipulating DNA fragments are described in “Current Protocols in Molecular Biology” Volumes 1 and 2, Ausubel F. M. et al., published by Greene Publishing Associates and Wiley Interscience (1989) or in Molecular cloning, T. Maniatis, E. F. Fritsch, J. Sambrook, 1982, or in Sambrook J. and Russell D., 2001, Molecular Cloning: a laboratory manual (Third edition)

### Example 1: Preparation of Mutated HPPD

### General Outline

The Arabidopsis thaliana AtHPPD coding sequence (1335 bp)(Genebank AF047834; WO 96/38567) was initially cloned into the expression vector pQE-30 (QIAGEN) in between the restriction sites of BamHI and HindIII.

The Pseudomonas fluorescens PfHPPD coding sequence (1174 bp) (Rüetschi et al., Eur. J. Biochem., 205, 459-466, 1992, WO 96/38567) was initially cloned into the unique NcoI site of the expression vector pKK233-2 (Pharmacia) that provides a start codon.

The vectors pQE-30-AtHPPD and pKK233-2-PfHPPD were used for PCR-mediated attachment of an NcoI restriction site and of a sequence encoding an N-terminal His₆-Tag to the 5′ ends and an XbaI restriction site to the 3′ ends of AtHPPD and PfHPPD.

The PCR product of the AtHPPD gene was isolated from an agarose gel, cut with the restriction enzymes NcoI and XbaI, purified with the MinElute™ PCR Purification Kit (Qiagen) and cloned into the pSE420(RI)NX vector cut with the same restriction enzymes.

Concerning the PfHPPD gene, the PCR product was isolated from an agarose gel and cloned into the pCR® 2.1-TOPO® vector. It was excised from this vector with the restriction enzymes NcoI and XbaI, isolated from an agarose gel and cloned into the pSE420(RI)NX vector cut with the same restriction enzymes.

Both pSE420(RI)NX-AtHPPD and -PfHPPD were then subjected to PCR-mediated site-directed mutagenesis to alter a defined codon at corresponding sites of both genes. The respective codon encodes Gly336 in WT PfHPPD and Gly422 in WT AtHPPD.

The mutated codons in the coding sequences are analyzed using the Pyrosequencing® technique.

PCR-mediated attachment of a sequence encoding an N-terminal His₆-tag and NcoI and XbaI restriction sites: The PCR reaction for each gene (AtHPPD and PfHPPD) was carried out in 24 wells of a 96 well PCR plate, respectively. Since the forward and reverse primers for this reaction differ in size by 18 (AtHPPD) and 22 bp (PfHPPD), an annealing temperature gradient from 40.9° C. to 64.5° C. was performed, each well being subjected to another annealing temperature within this range. When the primers anneal to the single stranded template for the first time, a 5′ overhang was produced in the new strand until its complementary strand is synthesized and this overhang formed by the 5′ region of the first primer is part of the template. The coding sequences were thereby extended at both ends, introducing a sequence encoding a N-terminal His₆-tag and a restriction site at both ends.

The reaction mixtures contain 500 ng of pQE-30-AtHPPD DNA (1 μL from plasmid maxipreparation) or 1 μg of pKK233-2-PfHPPD DNA (0.75 μL from plasmid maxipreparation), 1 μl of kerfi001 and kerfi002, respectively, for AtHPPD or kerfi003 and kerfi004, respectively, for PfHPPD (all primer solutions have a concentration of 10 pmol*μL⁻¹), 25 μl HotStarTaq Master Mix (Qiagen) and HyPure™ Molecular Biology Grade Water to a final volume of 50 μL. The PCR programme is set as follows:

### 1. 95° C. 15 min

### 2. 94° C. 30 s

40.9° C.−60.4° C. 30 s
72° C. 3 min
   Step 2 is repeated 20 times.

### 3. 72° C. 10 min

| Primer | |
|---|---|
| name | Primer sequence |
| kerfi001 | 5′-CCATGGCTCATCACCATCACCATCACCAAAACGCCG |
| | CCGTTTCAG-3′ |
| kerfi002 | 5′-TCTAGATCATCCCACTAACTGTTTGGC-3′ |
| kerfi003 | 5′- |
| | CCATGGCTCATCACCATCACCATCACGCAGATCTATACG |
| | AAAACCCAATGG-3′ |
| kerfi004 | 5′-TCTAGATTAATCGGCGGTCAATACACCAC-3′ |

The PCR reactions were subjected to agarose gel electrophoresis which all produced clear bands corresponding to fragments of approximately 1500 bp (AtHPPD) or 1100 bp (PfHPPD). The bands were excised from the gel and DNA was purified using the QIAquick® Gel Extraction Kit (Qiagen).

Cloning into pCR®2.1-TOPO® Vector (Invitrogen)

pCR® 2.1-TOPO® vector (3931 bp) was used for one-step cloning of Taq polymerase-amplified PCR products which display a 3′-adenosine (A) overhangs. The vector, in turn, was linearized and displayed single 3′-thymidine (T) overhangs at its ends. Topoisomerase I was covalently attached to these 3′-thymidines which served to covalently link the vector to the PCR product. For selection of bacterial cells carrying the vector, either ampicillin or kanamycin could be used. The vector possessed an XbaI restriction site within its multiple cloning site and an NcoI restriction site within the KanR gene.

DNA solutions obtained from each gel extraction were used for TOPO TA cloning, respectively. After transformation of E. coli TOP10 cells, each reaction yielded three white colonies (A1-A3, P1-P3) that were used to inoculate 5 mL LB/amp medium.

To determine whether the vectors of these colonies carried the correct inserted fragment, plasmid DNA was prepared from 4 mL of pCR®2.1-TOPO®-AtHPPD cultures A1-A3 and -PfHPPD cultures P1-P3 using the QIAprep® Spin Miniprep Kit (Qiagen). DNA solutions obtained from these plasmid preparations were subjected to a restriction digest with HindIII and XhoI which was then analyzed on a 1% agarose gel. Both HindIII and XhoI each possess a single restriction site in the pCR®2.1-TOPO®-AtHPPD/-PfHPPD vector, respectively. The restriction digest of DNA from clone A1 produced the expected bands representing a 1461 bp fragment (AtHPPD coding sequence) and the 3831 bp vector fragment; the restriction digest of P3 produced the expected bands representing a 1206 bp fragment (PfHPPD coding sequence) and the 3831 bp vector fragment on the agarose gel.

DNA obtained from plasmid maxipreparation using the QIAfilter™ Maxi Kit (Qiagen) and subsequent NaAc/EtOH precipitation from 100 mL of Al (AtHPPD) or P3 (PfHPPD) liquid LB/amp culture was used to determine the DNA sequence of the respective inserted HPPD gene in the pCR®2.1-TOPO® vector. DNA sequencing was carried out with the primers M13 uni (−21) and M13 rev (−29) by Eurofins MWG GmbH. Sequencing confirmed the correct DNA sequence of both AtHPPD and PfHPPD in the pCR®2.1-TOPO® vector, including the restriction sites at both ends of the coding sequences.

Cloning into pSE420(RI)NX

The cloning and expression vector pSE420(RI)NX (5261 bp) is based on the plasmid pSE420 by Invitrogen. Modifications of this vector include the addition of a kanamycin tolerance gene and the removal of the majority of the superlinker region (multiple cloning site).

The plasmid possesses the trp-lac (trc) promoter and the lacI^{q} gene that provides the lac repressor in every E. coli host strain. The lac repressor binds to the lac operator (lacO) and restricts expression of the target gene; this inhibition can be alleviated by induction with Isopropyl β-D-1-thiogalactopyranoside (IPTG).

The genes AtHPPD and PfHPPD were cloned into the vector pSE420(RI)NX in between the restriction sites of NcoI and XbaI.

PCR-Based Site-Directed Mutagenesis:

Template DNA (pSE420(RI)NX-AtHPPD and pSE420(RI)NX-PfHPPD) were isolated from E. coli TOP10 liquid culture by performing a plasmid minipreparation. The DNA solutions obtained from these minipreparations were diluted to a concentration of 0.05 μg*μL⁻¹.

PCR-based site-directed mutagenesis requires two chemically synthesized DNA primers (forward and reverse primer) that are complementary to the same DNA region, each of them to one strand of the double-stranded DNA template. These primers contain the desired mutation at their centre and cover a region of about 20-30 nucleotides of the template, including the mutation site and 10-15 bases on each of its sides. The mutation site covers three nucleotides that vary independently in the primers in order to obtain each possible codon at the selected site.

In circular PCR mutagenesis a plasmid template is completely copied by rolling circle replication starting from the 3′ OH end of a primer that is incorporated into the growing strand. Each new DNA molecule then carries one or more altered nucleotides that were contained in the primer. A high fidelity DNA polymerase is used in order to reduce the possibility of further undesired mutations.

The oligonucleotide primer pairs kerfi007/kerfi008 (AtHPPD) and kerfi011/kerfi012 (PfHPPD) were dissolved in water to a concentration of 10 pmol*μL⁻¹. For the mutagenesis PCR reaction, 50 ng of template plasmid from pSE420(RI)NX-AtHPPD or pSE420(RI)NX-PfHPPD minipreparations, diluted to a concentration of 0.05 μg*μL⁻¹, were used. The reaction mixture was composed as follows:

1 μL template plasmid (0.05 μg*μL⁻¹)

1.5 μL primer kerfi007 (or kerfi011) (10 pmol*μL⁻¹)
1.5 μL primer kerfi008 (or kerfi012) (10 pmol*μL⁻¹)

5 μL 10× reaction buffer

1 μL dNTP mix

40 μL HyPure™ Molecular Biology Grade Water

1 μL PfuUltra® High-Fidelity DNA polymerase (2.5 U*μL⁻¹)

The PCR programme was the same for mutagenesis of AtHPPD and PfHPPD and the elongation time was set to 7 minutes, assuming that it takes 1 minute to replicate 1 kb of plasmid DNA.

### 1. 95° C. 30 s

### 2. 95° C. 30 s

55° C. 30 s
68° C. 7 min
Step 2 is repeated 18 times.
   After the PCR reaction, the reactions were set on ice to cool down to room temperature.

| Primer | |
|---|---|
| name | Primer sequence |
| kerfi007 | 5′-GGTGGTTTTGGCAAANNNAATTTCTCTGAGCTC-3′ |
| kerfi008 | 5′-GAGCTCAGAGAAATTNNNTTTGCCAAAACCACC-3′ |
| kerfi011 | 5′-CAGCGCCTTGAAGTTNNNCTCGCCAAACCCATC-3′ |
| kerfi012 | 5′-GATGGGTTTGGCGAGNNNAACTTCAAGGCGCTG-3′ |

After the PCR reaction mutant plasmids were selected using the Dpn I restriction endonuclease. Only dam-methylated DNA is degraded by the restriction enzyme Dpn I whose restriction site G^{Me6}ATC is relatively abundant. Template plasmids which were produced by bacteria have been methylated and are therefore degraded. PCR-amplified DNA, however, remains intact.

1 μL of Dpn I restriction enzyme (10 U*μL⁻¹) was added to the PCR reactions and the solutions were mixed by pipetting up and down. After 1 minute of centrifugation (13,200 rpm) the reactions were incubated at 37° C. for 1 hour.

Mutant plasmids contained staggered nicks at the 5′ end of each primer and could be directly transformed into competent cells.

To concentrate mutant plasmids, a NaAc/EtOH precipitation was carried out and the DNA was resuspended in 10 μL of HyPure™ Molecular Biology Grade Water. 3 μL of these plasmid solutions were later used for transformation of electro competent E. coli K-12 MG1655 cells, and, in the case of AtHPPD, 1 μL was used for transformation of electro competent E. coli TOP10 cells.

For AtHPPD, a total of 62 E. coli K-12 MG1655 clones were obtained and cultivated for subsequent analysis of the mutated codon in Costar® 96 well 2 mL deep well plates. To obtain higher numbers of clones, E. coli TOP10 was used as an alternative host for cloning of mutagenized plasmids. Transformation of E. coli TOP10 cells with mutagenized plasmids yielded several hundreds of clones.

Concerning PfHPPD, a total of 252 E. coli K-12 MG1655 clones were obtained and cultivated for analysis as described for clones transformed with AtHPPD plasmids

### Example 2: Pyrosequencing® Reactions for Verifying Point Mutations

The Pyrosequencing® technology was used to verify point mutations by determining the nucleotide sequence of a short, defined section of DNA. A PCR reaction was performed first to amplify a short DNA fragment containing the section to be sequenced. The PCR-amplified template needs to be single-stranded and covalently attached to a biotin molecule at its 5′ end. Biotin served to attach the template non-covalently to streptavidin which was attached to a stationary phase of cross-linked agarose (sepharose).

Amplification of biotinylated DNA fragments: The PCR reaction was carried out in 96 well PCR plates. The reaction mixture contains 1 μL of forward primer solution (kerfi016 for AtHPPD, kerfi020 for PfHPPD; 10 pmol*μL⁻¹), 1 μL of reverse primer solution (contain a biotin modification at their 5′ ends; kerfi019 for AtHPPD, kerfi014 for PfHPPD; 10 pmol*μL⁻¹), 2 μL of liquid bacterial culture of a clone cultivated in a deepwell plate, 25 μL of HotStarTaq® Master Mix and 21 μL of HyPure™ Molecular Biology Grade Water.

The PCR programmes for AtHPPD and PfHPPD differed concerning the annealing temperatures which were set to 55° C. and 60° C., respectively.

### 1. 95° C. 15 min

### 2. 94° C. 30 s

55° C./60° C. 30 s
72° C. 30 s
   Step 2 was repeated 32 times.

### 3. 72° C. 10 min

| Primer | |
|---|---|
| name | Primer sequence |
| kerfi014 | 5′-GATCTTCTCGGAAACCCTGATG-3′ |
| (5′bio) | |
| kerfi016 | 5′-GGGATTCTTGTAGACAGAGATG-3′ |
| kerfi019 | 5′-CCCACTAACTGTTTGGCTTC-3′ |
| (5′bio) | |
| kerfi020 | 5′-GGCGGTCAATACACCACGAC-3′ |

Pyrosequencing® reaction: the Pyrosequencing® reaction (Biotage) was carried out in 96 well plates. To each 45 μL PCR reaction, 40 μL of Binding Buffer (10 mM Tris-HCl; 2 M NaCl; 1 mM EDTA; 0.1% Tween 20), 3 μL streptavidin sepharose beads (composition proprietary—GE Healthcare BioScience AB) and 12 μL ddH₂O were added. These mixtures were shaken for 10 minutes in the 96 well PCR plate.

With a “vacuum prep tool” each solution was then drawn through a small filter attached to a small metal tube, while the streptavidin beads, now bound to the biotinylated PCR product, were retained on the filters by the suction. According to this principle, the filters were then immersed in 70% ethanol for 5 seconds to wash the DNA and remove primers, dNTPs and other components of the PCR reaction. The procedure was repeated with 0.2 M NaOH to denature dsDNA and to leave only the biotinylated DNA strand bound to the streptavidin beads. After a final washing of the DNA in Washing Buffer, the “vacuum prep tool” was held over a PSQ™ 96 plate that contained 40 μL of Annealing Buffer and 0.1 μL of Pyrosequencing® primer solution (100 pmol*μL*; kerfi018 for AtHPPD/kerfi015 for PfHPPD) per well. The vacuum was then shut off and each filter was dipped into its corresponding well to dissolve the DNA that was retained by the filter. The plate was then incubated at 80° C. for 2 min to resolve secondary structures eventually formed within the DNA templates. While the solutions cooled to room temperature the Pyrosequencing® primers hybridized to their binding sites on the template.

The remaining components of the Pyrosequencing® reactions (620 μL of enzyme mixture, 620 μL of substrate mixture and 130 μL of each dNTP solution) were filled into separate wells of a cartridge. The cartridge and the PSQ™ plate were then placed inside the PyroMark™ ID.

The Pyrosequencing® instrument automatically added enzyme and substrate to the reaction mixture before the sequencing reaction is started by addition of the first dNTP. To determine the DNA sequence downstream of the primer, a SQA-run is conducted. The order of nucleotides added to the reaction mixture is defined in advance. The PyroMark™ ID software can be used to translate the Pyrogram® traces into the DNA sequence.

### Results:

The PCR-amplified fragment of AtHPPD has a size of 239 bp and the biotin is attached to the non-coding strand; the PfHPPD fragment comprises 142 bp and the biotin is attached to the coding strand.

The mutated codon in AtHPPD is located three bases downstream of the kerfi018 primer sequence. The first three bases sequenced are adenines, followed by the mutated codon. The coding strand of the AtHPPD fragment is synthesized by the DNA polymerase, so the sequence could be directly translated into the amino acid sequence.

Screening of 438 AtHPPD colonies issued 146 mutant genes, 181 wild type genes (codon GGC at position 422) and 111 failed sequencing reactions or ambiguous results.

The production of mutant clones by transformation of mutant plasmids in either E. coli K-12 MG1655 or E. coli TOP10 was therefore successful in 33% of all cases. Codons encoding all amino acids except lysine could be obtained. The genes containing the codons for glutamic acid, histidine, isoleucine, threonine, tryptophan and tyrosine were present in E. coli TOP10 clones from which DNA was prepared and transformed into E. coli K-12 MG1655 cells. If possible, synonymous codons were selected considering codon usage in E. coli K-12. No codon used at a frequency lower than 10% was chosen, most selected codons are used at a frequency higher than 35% (Codon usage database; E. coli K-12: http://www.kazusa.or.jp/codon/cgi-bin/showcodon.cgi?species=83333).

Starting from the primer kerfi015, the non-coding strand of the PfHPPD fragment is synthesized by the DNA polymerase, so the nucleotide sequence needed to be translated into the reverse complement before it could be translated into the amino acid sequence. The mutated codon immediately succeeds the primer and is therefore represented by the first three bases sequenced in the reaction.

Screening of 252 PfHPPD colonies issued 119 mutant genes, 73 unaltered genes (codon TGG at position 336) and 60 failed sequencing reactions or ambiguous results.

The production of mutant clones by transformation of mutant plasmids in E. coli K-12 MG1655 cells was therefore successful in 47% of all cases. Codons encoding all amino acids except alanine could be obtained. If possible, synonymous codons were selected considering codon usage in E. coli K-12 as described above for AtHPPD codons.

| Primer | |
|---|---|
| name | Primer sequence |
| kerfi015 | 5′-GACTCGAACAGCGCCTTGAAGTT-3′ |
| kerfi018 | 5′-GGATGTGGTGGTTTTGGC-3′ |

### Example 3: Assay for HPPD Activity

HPPD produces homogentisate and CO₂ from 4-HPP and O₂. The enzyme is incubated with its substrate 4-HPP in the presence or absence of an inhibitor. L-ascorbic acid is present as a reductant to retain the active site iron in the ferrous form and Catalase is present to degrade toxic H₂O₂. After an incubation time of one hour, the reaction is stopped by addition of 2,4-Dinitrophenylhydrazine (DNP). DNP forms a hydrazone derivative with the remaining 4-HPP molecules in the assay mixture which appears in an amber-brown colour at an alkaline pH. The amount of unconsumed 4-HPP is measured photometrically at 405 nm.

For preparation of inhibitor stock solutions, Tembotrione (M_{w}=440.82) and DKN (M_{w}=359.3) are dissolved in DMSO to a concentration of 10 mM. This stock solution is first diluted 20-fold in 25% DMSO to a concentration of 0.5 mM. Further dilutions are made with ddH₂O to obtain the inhibitor solutions used in the assay (5 μM, 10 μM and 20 μM). The respective inhibitor solution accounts for half of the assay mixture volume, meaning that its active concentration is again reduced 2-fold. This results in inhibitor concentrations of 2.5 μM, 5 μM and 10 μM. A 2% DMSO solution provides for half of the assay mixture in uninhibited reactions to normalize a possible inhibiting effect of DMSO.

The assay is designed for a HPPD concentration of 444 nM on a monomeric basis and a 4-HPP concentration of 500 μM. This corresponds to 44.4 pmol HPPD and 50 nmol 4-HPP in a 100 μL-assay mixture, resulting in an approximate 1000-fold excess of substrate in relation to the enzyme. The calculated theoretical molecular weight of an AtHPPD subunit is 49.515 kD which results in 2.2 μg HPPD per assay mixture. The calculated theoretical molecular weight of a PfHPPD subunit is 41.205 kD, resulting in 1.8 μg HPPD per assay mixture. The enzyme solution provides for one quarter of the assay mixture volume, so enzyme stock solutions are produced by diluting AtHPPD solutions to 88 μg*mL⁻¹ with 50 mM TRIS buffer; PfHPPD solutions are diluted to 72 μg*mL⁻¹.

The inhibitor concentrations (2.5 μM, 5 μM and 10 μM) provide for 5-fold, 10-fold and 20-fold excess of inhibitor compared to the amount of enzyme. A buffer/substrate solution is prepared which provides for one quarter of the assay mixture. 2.5 mL of buffer/substrate solution contain 1 mL 1 M TRIS buffer, 500 μL 10 mM 4-HPP solution, 500 μL 200 mM L-ascorbic acid solution, 13 μL Catalase solution and 487 μL ddH₂O. The assay is carried out in Greiner F-bottom 96 well microplates and all reactions are carried out as triplicates. The controls are carried out sixfold per plate and contain either 25 μL 50 mM TRIS instead of HPPD solution (corresponding to 0% consumption of 4-HPP) or a buffer/substrate solution that contains 500 μL 1 M TRIS instead of 500 μL 10 mM 4-HPP (corresponding to 100% consumption of HPP). The reaction is started by addition of 25 μL HPPD solution to a mixture of 50 μL of the respective inhibitor solution or 50 μL 2% DMSO and 25 μL buffer/substrate solution. The reaction is allowed to proceed for 1 h at room temperature. The reaction is stopped and coloration of 4-HPP is induced by addition of 50 μL 0.04% DNP/3.8 N HCl solution. After 15 min, addition of 100 μL 5 N KOH leads to the colour shift of the hydrazone derivative. Photometric measurement with a BMG FLUOstar Galaxy microplate reader is carried out immediately at 405 nm and data obtained is used for analysis of HPPD activities in presence and absence of an inhibitor.

### Results:

The AtHPPD mutants in position 422 with reference to the amino acid sequence of the Aradiposis HPPD of SEQ ID NO4 (i.e. Gly422Ala, -Arg, -Asn, -Asp, -Cys, -Glu, -His, -Leu, -Met, -Phe, -Pro, -Ser, -Tyr, and -Val) were tested along with the WT enzyme in the assay for HPPD activity (it is noted that Gly422 with reference to the amino acid sequence of the Aradiposis HPPD of SEQ ID NO4 corresponds to Gly336 with respect to the Pseudomonas reference sequence of SEQ ID NO: 2). All enzymes were active, but only the activities of the mutants Gly422Ala, -Asn, -Asp, -Cys, -His, -Met, -Phe, -Tyr and -Val were within or above the range 70%) of the WT enzyme. The WT enzyme retained 35% of its activity in the presence of 2.5 μM Tembotrione; only the mutants Gly422Asn, -Cys, -His and -Val retained higher activities ranging at 39, 44, 51 and 43%, respectively. Activities were further reduced at higher concentrations of Tembotrione. Only the mutant Gly422His displayed a residual activity of about 40% in the presence of 5 and 10 μM Tembotrione while all other enzymes displayed activities comparable to the WT enzyme at these inhibitor concentrations, ranging at approximately 20 and 10%, respectively (Table 1).

The PfHPPD mutants Gly336Arg, -Asp, -Gln, -Glu, His, -Leu, -Lys, -Met, -Phe, Thr, Trp and -Pro were tested along with the WT enzyme. With exception of the Gly336Pro mutant, whose uninhibited activity ranged below 70% of WT activity, the activities of the Gly336 mutants were within or above the range of the WT enzyme (≥75%). The WT enzyme retained only 5% of its activity in the presence of 2.5 μM Tembotrione while the mutants Gly336Asp, -Arg, -Gln, -Glu, -His, -Met, -Phe and -Trp retained activities above 14%. The highest residual activities were those of Gly336His (26%) and Gly336Phe (33%). Interestingly, the Gly336His mutant displayed residual activities of 13 and 11.2% in the presence of 5 and 10 μM Tembotrione, respectively, while the activities of Gly336Phe was reduced to 12.4 and 2.5%, respectively. The Gly336Met mutant, displayed residual activities of 7 and 10% respectively at these inhibitor concentrations, while the activity of the WT enzyme was reduced to zero. (Table 1).

TABLE 1
Relative activity (in percentage) of Pf HPPD
and At HPPD mutants in presence and absence of
Tembotrione; Activities are normalized by setting the
uninhibited enzyme activity to 100%
Pseudomonas fluorescens HPPD

| | |
|---|---|
| | Concentration of |
| Gly336 | Tembotrione (μM) |

| mutant | 0 | 2.5 | 5 | 10 |
|---|---|---|---|---|
| Arg | 100 | 14 | 7 | 2 |
| Asp | 100 | 18 | 9 | 0 |
| Gln | 100 | 14 | 0 | 0 |
| Glu | 100 | 15 | 7 | 0 |
| Gly | 100 | 5 | 0 | 0 |
| His | 100 | 26 | 13 | 11 |
| Leu | 100 | 4 | 0 | 0 |
| Lys | 100 | 6 | 0 | 0 |
| Met | 100 | 16 | 7 | 10 |
| Phe | 100 | 33 | 12 | 3 |
| Pro | 100 | 5 | 4 | 0 |
| Thr | 100 | 8 | 2 | 2 |
| Trp | 100 | 21 | 7 | 0 |

Arabidopsis thaliana HPPD

| | |
|---|---|
| | Concentration of |
| Gly422 | Tembotrione (μM) |

| mutant* | 0 | 2.5 | 5 | 10 |
|---|---|---|---|---|
| Ala | 100 | 25 | 21 | 15 |
| Arg | 100 | 17 | 1 | 1 |
| Asn | 100 | 39 | 26 | 15 |
| Asp | 100 | 20 | 7 | 10 |
| Cys | 100 | 44 | 27 | 19 |
| Glu | 100 | 24 | 24 | 0 |
| Gly | 100 | 35 | 21 | 12 |
| His | 100 | 50 | 31 | 40 |
| Leu | 100 | 31 | 23 | 14 |
| Met | 100 | 18 | 13 | 12 |
| Phe | 100 | 30 | 16 | 11 |
| Pro | 100 | 0 | 0 | 0 |
| Ser | 100 | 18 | 4 | 0 |
| Tyr | 100 | 26 | 11 | 0 |
| Val | 100 | 43 | 22 | 14 |

*Mutation at the gly in position 422 with reference to the amino acid sequence of the Aradiposis HPPD of SEQ ID NO4 (corresponds to Gly336 with reference to the amino acid sequence of the Pseudomonas HPPD of SEQ ID NO2)

### Example 4: Assay for PDH Activity

The prephenate dehydrogenase activity was measured at 25° C. by spectrophotometric monitoring at 340 nm of the formation of NADH or NADPH in a solution containing 50 mM of tris-HCl, pH 8.6, 300 μM of prephenate, and 1 mM of NAD or NADP in a total volume of 200 μl.

### Example 3: Construction of Chimeric Genes for the Evaluation of Unmutated and Mutated Pf HPPD in Tobacco

A) Construction of the Chimeric Genes:

The vector which is employed in order to make the constructs which HPPD (wild-type or mutants) to be expressed in type PBD6 tobacco plants is designated pRP-RD224. This vector was initially conceived for cloning all the Pseudomonas HPPD mutants by simply replacing the truncated HPPD gene of this vector between the KpnI and BstEII sites. Its construction from the binary vector pBI121 (Clontech) is extensively described in WO 99/24585.

Clone pRP-RD224 therefore has the following structure:
RB/Nos promoter/NPTII/Nos terminator/double histone promoter/tev/otp/truncated HPPD/Nos terminator/LB
   wherein “truncated HPPD” refers to the sequence encoding the Pf HPPD truncated of approximately 500 base pairs in order subsequently to facilitate screening of the transformed colonies which have integrated the mutant HPPDs (WO99/24585)

pRP-RD224 mutants: The DNAs of the vectors carrying the mutated and unmutated HPPDs were digested with KpnI and BstEII, purified and then ligated into vector pRP-RD224, which had been digested with KpnI and BstEII and purified. The transformants which had integrated the mutated HPPD gene were selected for the size of the insert by digesting with KpnI and BstEII. The resulting clones are designated pRP-RD224 to which is added the type of mutation which has been carried out on the HPPD; in this way, the following clones were created: pRP RD224 Pf (for the unmutated enzyme), pRP RD224 PfH336 (for the enzyme having a histidine at position 336), pRP RD224 PfM336 (for the enzyme having a methionine at position 336), and pRP RD224 PfF336 (for the enzyme having a phenylalanine at position 336).

### Example 4: Construction of a Chimeric Gene Overexpressing PDH

The construction of a chimeric gene overexpressing PDH comprises assembling, in the direction of transcription, a “double histone” promoter (PdH4) as described in patent application EP 0 507 698, the tobacco etch virus translational enhancer (TEV) sequence described in Carrington and Freed (1990), a sequence encoding an optimized transit peptide (OTP) as described in patent application EP 0 508 909, the coding portion of the yeast PDH gene described in Mannhaupt et al. (1989) and the nos terminator of the nopaline synthase gene described in Bevan et al. (1983). The assembly was then cloned into the binary vector pRD 224 containing a kanamycin tolerance gene(NPTII), to give the vector pRD 224-PDH.

This binary vector was then used to transform the Agrobacterium strain EHA 105 and to give the Agrobacterium strain EHA 105-pRD 224-PDH. This Agrobacterium strain was used to transform tobacco plants transformed with the chimeric genes as described in example 3.

The transformed plants are selected on kanamycin.

### CITED REFERENCES

Abou-Zeid et al., 1995, Applied Env Microb 41: 1298-1302
Ausubel F. M. et al., “Current Protocols in Molecular Biology” Volumes 1 and 2, published by Greene Publishing Associates and Wiley Interscience (1989)
Bevan et al., 1983, Nucleic Acids Res. 11(2), 369-385
Bonner et al., 1995, Plant Cells Physiol. 36, 1013-1022
Byng et al., 1981, Phytochemistry 6: 1289-1292
Carrington and Freed, 1990; J. Virol. 64: 1590-1597
Christou et al., 1991, Biotechnology 9:957
Connely and Conn, 1986, Z. Naturforsch 41c: 69-78
Crouch N. P. et al., 1997, Tetrahedron, 53, 20, 6993-7010
Datla, R. et al., 1997, Biotechnology Ann. Rev. 3, 269-296
Gaines et al., 1982, Plants 156: 233-240
Henner et al., 1986, Gene 49 (1) 147-152
Hiei et al., 1994, Plant J 6:271-282
Hiei et al., 1997, Plant Mol Biol. 35:205-21
Hudson et al., 1984, J. Mol. Biol. 180(4), 1023-1051
Fritze et al., 2004, Plant Physiology 134:1388-1400
Garcia et al., 1997, Biochem. J. 325, 761-769
Garcia et al., 1999, Plant Physiol. 119, 1507-1516
Gould J. H. and Magallanes-Cedeno M., 1998, Plant Molecular Biology reporter, 16:1-10
Horsch et al., 1985, Science 227: 1229-1231
Lingens et al., 1967, European J. Biochem 1: 363-374
Maniatis T., Fritsch E. F., in Molecular cloning, Sambrook, 1982.
Mannhaupt et al., 1989, Gene 85, 303-311
Matringe et al., 2005, Pest Management Science 61:269-276
Mitchell et al., 2001, Pest Management Science 57:120-128
Pallett et al., 2001, Pest Management Science 57:133-142
Sambrook et al., 1989, Molecular cloning: a laboratory manual, second edition, Cold Spring Harbor Laboratory Press, Cold Spring Harbor, N.Y.)
Sambrook J. and Russell D., 2001, Molecular Cloning: A laboratory Manual (third edition), ISBN 978-087969577-4 CSHL Press
Sampathkumar and Morrisson, 1982, Bioch Biophys Acta 701: 204-211
Sawahel W. A., 2001, Plant Molecular Biology reporter, 19:377a-377f
Schulz et al., 1993, FEBS Letters 318:162-166
Xia et al., 1992, J. Gen. Microbiol. 138(7), 1309-1316
Zapata C., 1999, theoretical Applied Genetics, 98(2):1432-2242

1. A mutated hydroxyphenylpyruvate dioxygenase (HPPD) which retains its properties of catalysing the conversion of para-hydroxyphenylpyruvate (HPP) to homogentisate and which is less sensitive to a HPPD inhibitor than the original unmutated HPPD, characterized in that it contains a mutation on the amino acid glycine in position 336 with reference to the amino acid sequence of the Pseudomonas HPPD of SEQ ID NO:2 which is selected from the following group: Gly336His, Gly336Met, Gly336Phe, and Gly336Cys, provided that when the mutation is Gly336His, the amino acid at position 334 with reference to the amino acid sequence of the Pseudomonas HPPD of SEQ ID NO:2 is Gly.

2. The mutated HPPD according to claim 1, characterized in that the mutated HPPD contains a second mutation.

3. The mutated HPPD according to claim 2, characterized in that the second mutated amino acid is selected from the selected amino acids: Pro215, Gly298, Gly332, Phe333, Gly334 and Asn337, with reference to the Pseudomonas HPPD sequence of SEQ ID NO:2.

4. A nucleic acid sequence which encodes a mutated HPPD according to claim 1.

5. A chimeric gene which comprises a coding sequence as well as heterologous regulatory element in the 5′ and optionally in the 3′ positions, which are able to function in a host organism, characterized in that the coding sequence contains at least a nucleic acid sequence according to claim 4.

6. The chimeric gene according to claim 5 characterized in that it contains in the 5′ position of the nucleic acid sequence which encodes a mutated HPPD, a nucleic acid sequence which encodes a plant transit peptide, with this sequence being arranged between the promoter region and the sequence encoding the mutated HPPD so as to permit expression of a transit peptide/mutated HPPD fusion protein.

7. A transit peptide/mutated HPPD fusion protein, with the mutated HPPD being defined according to claim 1.

8. A cloning and/or expression vector for transforming a host organism, characterized in that it contains at least one chimeric gene according to claim 5.

9. A plant cell, characterized in that it contains at least a nucleic acid sequence according to claim 4.

10. The plant cell according to claim 9 characterized in that it contains, in addition, a gene that is functional in plants allowing overexpression of a PDH (prephenate dehydrogenase) enzyme.

11. A transformed plant, characterized in that it contains a transformed plant cell according to claim 9.

12. A transformed seed, characterized in that it contains a transformed plant cell according to claim 9.

13. A method for obtaining a plant resistant to a HPDD inhibitor, characterized in that the plant is transformed with a chimeric gene according to claim 5.

14. A method for obtaining a plant resistant to a HPDD inhibitor according to claim 13, characterized in that the plant is further transformed, simultaneously or successively, with a second gene functional in this plant allowing overexpression of a PDH (prephenate dehydrogenase) enzyme.

15. A method for controlling weeds in an area or a field which contains transformed seeds according to claim 12, which method comprises applying, to the said area of the field, a dose of a HPPD inhibitor herbicide which is toxic for the said weeds, without significantly affecting said seeds.

16. A method for obtaining oil or meal comprising growing a transformed plant according to claim 11, optionally treating such plant with an HPPD inhibitor herbicide, harvesting the grains and milling the grains to make meal and optionally extract the oil.

17. The method according to claim 13, in which the HPPD inhibitor is a triketone HPPD inhibitor.

18. The method according to claim 17, in which the HPPD inhibitor is selected from tembotrione, mesotrione, and sulcotrione.

19. The mutated HPPD according to claim 1, characterized in that the mutation on the amino acid glycine in position 336 is Gly336His.

20. The mutated HPPD according to claim 1, characterized in that the mutation on the amino acid glycine in position 336 is Gly336Met.

21. The mutated HPPD according to claim 1, characterized in that the mutation on the amino acid glycine in position 336 is Gly336Phe.

22. The mutated HPPD according to claim 1, characterized in that the mutation on the amino acid glycine in position 336 is Gly336Cys.


SEQUENCE LISTING (32 sequences)

SEQ ID NO: 1 - DNA, 1077 bp, Pseudomonas fluorescens
  CDS (1)..(1077)
atggcagatctatacgaaaacccaatgggcctgatgggctttgaattcatcgaattcgcgtcgccgacgccgggtaccctggagccgatcttcgagatcatgggcttcaccaaagtcgcgacccaccgttccaagaacgtgcacctgtaccgccagggcgagatcaacctgatcctcaacaacgagcccaacagcatcgcctcctactttgcggccgaacacggcccgtcggtgtgcggcatggcgttccgcgtgaaggactcgcaaaaggcctacaaccgcgccctggaactcggcgcccagccgatccatattgacaccgggccgatggaattgaacctgccggcgatcaagggcatcggcggcgcgccgttgtacctgatcgaccgtttcggcgaaggcagctcgatctacgacatcgacttcgtgtacctcgaaggtgtggagcgcaatccggtcggtgcaggtctcaaagtcatcgaccacctgacccacaacgtctatcgcggccgcatggtctactgggccaacttctacgagaaattgttcaacttccgtgaagcgcgttacttcgatatcaagggcgagtacaccggcctgacttccaaggccatgagtgcgccggacggcatgatccgcatcccgctgaacgaagagtcgtccaagggcgcggggcagatcgaagagttcctgatgcagttcaacggcgaaggcatccagcacgtggcgttcctcaccgacgacctggtcaagacctgggacgcgttgaagaaaatcggcatgcgcttcatgaccgcgccgccagacacttattacgaaatgctcgaaggccgcctgcctgaccacggcgagccggtggatcaactgcaggcacgcggtatcctgctggacggatcttccgtggaaggcgacaaacgcctgctgctgcagatcttctcggaaaccctgatgggcccggtgttcttcgaattcatccagcgcaagggcgacgatgggtttggcgaggggaacttcaaggcgctgttcgagtccatcgaacgtgaccaggtgcgtcgtggtgtattgaccgccgattaa

SEQ ID NO: 2 - protein, 358 aa, Pseudomonas fluorescens
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGEGNFKALFESIERDQVRRGVLTAD

SEQ ID NO: 3 - DNA, 1338 bp, Arabidopsis thaliana
  CDS (1)..(1338)
atgggccaccaaaacgccgccgtttcagagaatcaaaaccatgatgacggcgctgcgtcgtcgccgggattcaagctcgtcggattttccaagttcgtaagaaagaatccaaagtctgataaattcaaggttaagcgcttccatcacatcgagttctggtgcggcgacgcaaccaacgtcgctcgtcgcttctcctggggtctggggatgagattctccgccaaatccgatctttccaccggaaacatggttcacgcctcttacctactcacctccggtgacctccgattccttttcactgctccttactctccgtctctctccgccggagagattaaaccgacaaccacagcttctatcccaagtttcgatcacggctcttgtcgttccttcttctcttcacatggtctcggtgttagagccgttgcgattgaagtagaagacgcagagtcagctttctccatcagtgtagctaatggcgctattccttcgtcgcctcctatcgtcctcaatgaagcagttacgatcgctgaggttaaactatacggcgatgttgttctccgatatgttagttacaaagcagaagataccgaaaaatccgaattcttgccagggttcgagcgtgtagaggatgcgtcgtcgttcccattggattatggtatccggcggcttgaccacgccgtgggaaacgttcctgagcttggtccggctttaacttatgtagcggggttcactggttttcaccaattcgcagagttcacagcagacgacgttggaaccgccgagagcggtttaaattcagcggtcctggctagcaatgatgaaatggttcttctaccgattaacgagccagtgcacggaacaaagaggaagagtcagattcagacgtatttggaacataacgaaggcgcagggctacaacatctggctctgatgagtgaagacatattcaggaccctgagagagatgaggaagaggagcagtattggaggattcgacttcatgccttctcctccgcctacttactaccagaatctcaagaaacgggtcggcgacgtgctcagcgatgatcagatcaaggagtgtgaggaattagggattcttgtagacagagatgatcaagggacgttgcttcaaatcttcacaaaaccactaggtgacaggccgacgatatttatagagataatccagagagtaggatgcatgatgaaagatgaggaagggaaggcttaccagagtggaggatgtggtggttttggcaaaggcaatttctctgagctcttcaagtccattgaagaatacgaaaagactcttgaagccaaacagttagtgggatga

SEQ ID NO: 4 - protein, 445 aa, Arabidopsis thaliana
MGHQNAAVSENQNHDDGAASSPGFKLVGFSKFVRKNPKSDKFKVKRFHHIEFWCGDATNVARRFSWGLGMRFSAKSDLSTGNMVHASYLLTSGDLRFLFTAPYSPSLSAGEIKPTTTASIPSFDHGSCRSFFSSHGLGVRAVAIEVEDAESAFSISVANGAIPSSPPIVLNEAVTIAEVKLYGDVVLRYVSYKAEDTEKSEFLPGFERVEDASSFPLDYGIRRLDHAVGNVPELGPALTYVAGFTGFHQFAEFTADDVGTAESGLNSAVLASNDEMVLLPINEPVHGTKRKSQIQTYLEHNEGAGLQHLALMSEDIFRTLREMRKRSSIGGFDFMPSPPPTYYQNLKKRVGDVLSDDQIKECEELGILVDRDDQGTLLQIFTKPLGDRPTIFIEIIQRVGCMMKDEEGKAYQSGGCGGFGKGNFSELFKSIEEYEKTLEAKQLVG

SEQ ID NO: 5 - DNA, 1182 bp, Mus musculus
  CDS (1)..(1182)
atgacaacctacaacaacaaaggaccaaagcctgagagaggccggttcctccatttccactcggtgaccttctgggttggcaatgccaagcaggctgcttccttctactgcaacaagatgggctttgaacctctggcctacaggggcctagagactggctcccgggaggtagtcagccacgtcatcaagcaagggaaaattgtgtttgttctctgctctgctctcaatccctggaacaaagagatgggcgaccacttggtgaagcatggcgacggggtgaaagacatcgcattcgaggtggaagactgcgaccacattgtgcagaaagctcgagaacggggcgccaaaattgtgcgggagccatgggtggagcaagacaaatttgggaaggtgaagtttgctgtgctgcagacgtatggagataccacacacaccctggtggagaagatcaactacactggccgtttcttacctggattcgaggccccaacatacaaggataccctgcttccaaaactacccagatgtaaccttgagatcattgaccacattgtaggcaaccaacccgaccaagaaatgcagtctgcctcagaatggtacctgaaaaacctgcagttccaccggttctggtccgtggacgacacgcaggtgcacacggagtacagctctctgcgctccattgtggtgaccaactacgaggaatccatcaaaatgcccatcaacgagccagctccgggcaggaagaagtctcagatccaggaatatgtggactataatgggggtgctggggtccagcacatcgctctcaagacggaagacatcatcacagcaatccgccacttgagggagcgaggcacggagttcttggccgccccatcttcttactacaaactgcttcgggagaatctcaagtcagccaagatccaggtgaaagagagcatggacgtcctggaggagctgcatatcctagtcgactatgacgagaaaggctacctcctacagatcttcaccaagcccatgcaggaccggcccacactcttcctggaagtcattcaacgtcacaaccaccagggctttggagcgggcaacttcaactctctgttcaaggcgttcgaggaggagcaagccctacggggcaacctcactgacctggagcccaatggtgtgaggtctggaatgtaa

SEQ ID NO: 6 - protein, 393 aa, Mus musculus
MTTYNNKGPKPERGRFLHFHSVTFWVGNAKQAASFYCNKMGFEPLAYRGLETGSREVVSHVIKQGKIVFVLCSALNPWNKEMGDHLVKHGDGVKDIAFEVEDCDHIVQKARERGAKIVREPWVEQDKFGKVKFAVLQTYGDTTHTLVEKINYTGRFLPGFEAPTYKDTLLPKLPRCNLEIIDHIVGNQPDQEMQSASEWYLKNLQFHRFWSVDDTQVHTEYSSLRSIVVTNYEESIKMPINEPAPGRKKSQIQEYVDYNGGAGVQHIALKTEDIITAIRHLRERGTEFLAAPSSYYKLLRENLKSAKIQVKESMDVLEELHILVDYDEKGYLLQIFTKPMQDRPTLFLEVIQRHNHQGFGAGNFNSLFKAFEEEQALRGNLTDLEPNGVRSGM

SEQ ID NO: 7 - DNA, 1200 bp, Coccidioides immitis
  CDS (1)..(1200)
atggcaccagccgctgactccccgacgcttcaacccgcccagccctctgatctcaatcagtatagaggatacgaccacgtccactggtatgtcggaaacgctaagcaggccgctacctactatgtcactcgcatgggtttcgagagagtagcctatcgcggattggagactggctccaaagcggtggcctcgcatgttgtgcgaaacggaaacatcaccttcatcttgacttcgccccttcgatccgttgagcaggcttctcgtttccccgaggacgaggctctcctgaaggagatccacgcccatctcgagagacacggcgatggtgtcaaggacgtcgccttcgaggtcgactgcgtagagtctgtcttctcggctgccgttaggaacggtgctgaggttgtttccgatgtcagaacggttgaagatgaggatggccagatcaagatggcgaccatccgaacttatggcgagaccactcacaccctcatcgaaagatccggctacaggggcggattcatgccgggataccggatggagagcaatgccgacgccacttccaagttccttccaaaggttgtgcttgagagaatagaccactgcgttggaaaccaggactgggacgagatggagcgagtctgcgactactacgagaagatcctcggattccaccgtttctggtccgttgatgacaaggacatctgcactgaattctctgcactgaagagtatcgtcatggcatctccaaatgatatcgtcaagatgcccatcaacgagcccgccaagggaaagaaacaatcccagattgaagaatatgttgacttctacaatggtgctggcgttcagcacattgctctccgaaccaacaacatcatcgatgccatcaccaacctcaaggcgcgcggcaccgaattcatcaaggttccagagacctactatgaagacatgaagattcgcctcaagagacaaggcctggtcctcgatgaggactttgagaccctgaagagcctggacatccttatcgactttgacgagaatgggtatctcctgcagcttttcaccaagcatctcatggatcgcccaaccgttttcattgaaatcatccaacgcaacaacttttccggtttcggtgcgggcaacttcagggccctcttcgaggctattgagcgtgagcaggctctccgtggcacccttatctag

SEQ ID NO: 8 - protein, 399 aa, Coccidioides immitis
MAPAADSPTLQPAQPSDLNQYRGYDHVHWYVGNAKQAATYYVTRMGFERVAYRGLETGSKAVASHVVRNGNITFILTSPLRSVEQASRFPEDEALLKEIHAHLERHGDGVKDVAFEVDCVESVFSAAVRNGAEVVSDVRTVEDEDGQIKMATIRTYGETTHTLIERSGYRGGFMPGYRMESNADATSKFLPKVVLERIDHCVGNQDWDEMERVCDYYEKILGFHRFWSVDDKDICTEFSALKSIVMASPNDIVKMPINEPAKGKKQSQIEEYVDFYNGAGVQHIALRTNNIIDAITNLKARGTEFIKVPETYYEDMKIRLKRQGLVLDEDFETLKSLDILIDFDENGYLLQLFTKHLMDRPTVFIEIIQRNNFSGFGAGNFRALFEAIEREQALRGTLI

SEQ ID NO: 9 - DNA, 1260 bp, Mycosphaerella graminicola
  CDS (1)..(1260)
atggcacccggagcactcctcgtcacatcacagaatggaagaacgagccccctctacgactccgatggctatgtaccagcgcctgcggctctagtagtaggtggtgaggtcaattacagaggctaccatcatgcagaatggtgggtgggcaatgcaaagcaggtggcgcaattctacatcacacgcatgggcttcgagcctgttgcacacaaaggtctggagaccggatctcgcttctttgccagccacgttgtccagaacaacggcgttcgcttcgtcttcacatcacctgttcggtcatcggcacggcaaacactcaaagcagcgcctctcgcggaccaagcacgcctcgacgaaatgtacgatcacctcgacaagcacggagatggagtgaaggatgttgccttcgaagttgacgatgtcttggctgtgtacgagaacgcagttgcgaatggtgcggagtccgtcagttcaccacataccgattcatgcgacgaaggcgatgtgatctccgcggcgatcaagacatacggagacaccacgcacactttcatccaacgcacaacatatacaggaccatttcttcctggctatcgatcatgtaccacagtggattcggccaacaagttcttgccaccagtcaatctcgaagcgatcgatcactgtgtcggcaatcaagactgggacgagatgagcgatgcctgcgacttctacgagcgctgtcttggattccatcgcttctggagtgtcgatgacaaggacatctgtacggagttctccgcgctgaagtctatcgttatgagttctcccaaccaggtagtcaagatgccaatcaacgagcccgcccatggcaagaagaagagccagatcgaggagtacgtcgatttctacaatggacctggcgtacaacacatcgctctccgtacgccaaacatcatcgaggcagtatcaaacttgcggtcaagaggcgtggagttcatcagcgtgccagatacgtactacgagaacatgcgtcttcgtctcaaagcggcaggaatgaagctggaggagtcattcgacatcattcaaaagctgaacatcctcatcgatttcgacgaaggtggctatttgctgcagctgttcacgaagccgctgatggatcggccgacggtcttcattgaaatcattcaacggaacaactttgatggcttcggagctggaaacttcaagagtctgttcgaggcgattgagcgagagcaggacttgcgtggcaatctctag

SEQ ID NO: 10 - protein, 419 aa, Mycosphaerella graminicola
MAPGALLVTSQNGRTSPLYDSDGYVPAPAALVVGGEVNYRGYHHAEWWVGNAKQVAQFYITRMGFEPVAHKGLETGSRFFASHVVQNNGVRFVFTSPVRSSARQTLKAAPLADQARLDEMYDHLDKHGDGVKDVAFEVDDVLAVYENAVANGAESVSSPHTDSCDEGDVISAAIKTYGDTTHTFIQRTTYTGPFLPGYRSCTTVDSANKFLPPVNLEAIDHCVGNQDWDEMSDACDFYERCLGFHRFWSVDDKDICTEFSALKSIVMSSPNQVVKMPINEPAHGKKKSQIEEYVDFYNGPGVQHIALRTPNIIEAVSNLRSRGVEFISVPDTYYENMRLRLKAAGMKLEESFDIIQKLNILIDFDEGGYLLQLFTKPLMDRPTVFIEIIQRNNFDGFGAGNFKSLFEAIEREQDLRGNL

SEQ ID NO: 11 - DNA, 1305 bp, Hordeum vulgare
  CDS (1)..(1305)
atgccgcccacccccaccacccccgcggctaccggcgccgccgccgcggtgacgccggagcacgcgcgaccgcaccgaatggtccgcttcaacccgcgcagcgaccgcttccacacgctctccttccaccacgtcgagttctggtgcgcggacgccgcctccgccgccggccgcttcgcgttcgcgctcggcgcgccgctcgccgccaggtccgacctctccacggggaactccgcgcacgcctcccagctgctccgctcgggctccctcgccttcctcttcaccgcgccctacgccaacggctgcgacgccgccaccgcctccctgccctccttctccgccgacgccgcgcgccggttctccgccgaccacgggatcgcggtgcgctccgtagcgctgcgcgtcgcagacgccgccgaggccttccgcgccagtcgtcgacggggcgcgcgcccggccttcgcccccgtggacctcggccgcggcttcgcgttcgcggaggtcgagctctacggcgacgtcgtgctccgcttcgtcagccacccggacggcacggacgtgcccttcttgccggggttcgagggcgtaaccaacccggacgccgtggactacggcctgacgcggttcgaccacgtcgtcggcaacgtcccggagcttgcccccgccgcagcctacatcgccgggttcacggggttccacgagttcgccgagttcacggcggaggacgtgggcacgaccgagagcgggctcaactcggtggtgctcgccaacaactcggagggcgtgctgctgccgctcaacgagccggtgcacggcaccaagcgccggagccagatacagacgttcctggaacaccacggcggcccgggcgtgcagcacatcgcggtggccagcagtgacgtgctcaggacgctcaggaagatgcgtgcgcgctccgccatgggcggcttcgacttcctgccacccccgctgccgaagtactacgaaggcgtgcgacgccttgccggggatgtcctctcggaggcgcagatcaaggaatgccaggagctgggtgtgctcgtcgatagggacgaccaaggggtgttgctccaaatcttcaccaagccagtaggggacaggccgaccttgttcctggagatgatccagaggatcgggtgcatggagaaggacgagagaggggaagagtaccagaagggtggctgcggcgggttcggcaaaggcaacttctccgagctgttcaagtccattgaagattacgagaagtcccttgaagccaagcaatctgctgcagttcagggatcatag

SEQ ID NO: 12 - protein, 434 aa, Hordeum vulgare
MPPTPTTPAATGAAAAVTPEHARPHRMVRFNPRSDRFHTLSFHHVEFWCADAASAAGRFAFALGAPLAARSDLSTGNSAHASQLLRSGSLAFLFTAPYANGCDAATASLPSFSADAARRFSADHGIAVRSVALRVADAAEAFRASRRRGARPAFAPVDLGRGFAFAEVELYGDVVLRFVSHPDGTDVPFLPGFEGVTNPDAVDYGLTRFDHVVGNVPELAPAAAYIAGFTGFHEFAEFTAEDVGTTESGLNSVVLANNSEGVLLPLNEPVHGTKRRSQIQTFLEHHGGPGVQHIAVASSDVLRTLRKMRARSAMGGFDFLPPPLPKYYEGVRRLAGDVLSEAQIKECQELGVLVDRDDQGVLLQIFTKPVGDRPTLFLEMIQRIGCMEKDERGEEYQKGGCGGFGKGNFSELFKSIEDYEKSLEAKQSAAVQGS

SEQ ID NO: 13 - DNA, 1332 bp, Zea mays
  CDS (1)..(1332)
atgcccccgacccccacagccgccgcagccggcgccgccgtggcggcggcatcagcagcggagcaagcggcgttccgcctcgtgggccaccgcaacttcgtccgcttcaacccgcgctccgaccgcttccacacgctcgcgttccaccacgtggagctctggtgcgccgacgcggcctccgccgcgggccgcttctccttcggcctgggcgcgccgctcgccgcacgctccgacctctccacgggcaactccgcgcacgcgtccctgctgctccgctccggctccctctccttcctcttcacggcgccctacgcgcacggcgccgacgctgccaccgccgcgctgccctccttctccgccgccgccgcgcggcgcttcgcagccgaccacggcctcgcggtgcgcgccgtcgcgctccgcgtcgccgacgccgaggacgccttccgcgccagcgtcgcggccggggcgcgcccggcgttcggccccgtcgacctcggccgcggcttccgcctcgccgaggtcgagctctacggcgacgtcgtgctccggtacgtgagctacccggacggcgccgcgggcgagcccttcctgccggggttcgagggcgtggccagccccggggcggccgactacgggctgagcaggttcgaccacatcgtcggcaacgtgccggagctggcgcccgccgccgcctacttcgccggcttcacggggttccacgagttcgccgagttcacgacggaggacgtgggcaccgcggagagcggcctcaactccatggtgctcgccaacaactcggagaacgtgctgctcccgctcaacgagccggtgcacggcaccaagcgccgcagccagatacaaacgttcctggaccaccacggcggccccggcgtgcagcacatggcgctggccagcgacgacgtgctcaggacgctgagggagatgcaggcgcgctcggccatgggcggcttcgagttcatggcgcctcccacatccgactactatgacggcgtgaggcggcgcgccggggacgtgctcacggaagcacagattaaggagtgccaggagctaggggtgctggtggacagggatgaccagggcgtgctgctccaaatcttcaccaagccagtgggggacaggccaacgctgttcttggaaatcatccaaaggatcgggtgcatggagaaggatgagaaggggcaagaataccaaaagggtggctgcggcgggttcggcaagggaaacttctcgcagctgttcaagtccatcgaggattatgagaagtcccttgaagccaagcaagctgctgcagcagctgcagctcagggatcc

SEQ ID NO: 14 - protein, 444 aa, Zea mays
MPPTPTAAAAGAAVAAASAAEQAAFRLVGHRNFVRFNPRSDRFHTLAFHHVELWCADAASAAGRFSFGLGAPLAARSDLSTGNSAHASLLLRSGSLSFLFTAPYAHGADAATAALPSFSAAAARRFAADHGLAVRAVALRVADAEDAFRASVAAGARPAFGPVDLGRGFRLAEVELYGDVVLRYVSYPDGAAGEPFLPGFEGVASPGAADYGLSRFDHIVGNVPELAPAAAYFAGFTGFHEFAEFTTEDVGTAESGLNSMVLANNSENVLLPLNEPVHGTKRRSQIQTFLDHHGGPGVQHMALASDDVLRTLREMQARSAMGGFEFMAPPTSDYYDGVRRRAGDVLTEAQIKECQELGVLVDRDDQGVLLQIFTKPVGDRPTLFLEIIQRIGCMEKDEKGQEYQKGGCGGFGKGNFSQLFKSIEDYEKSLEAKQAAAAAAAQGS

SEQ ID NO: 15 - DNA, 1329 bp, Daucus carota
  CDS (1)..(1329)
atggggaaaaaacaatcggaagctgaaattctctcaagcaattcatcaaacacctctcctgcaacattcaagctggtcggtttcaacaacttcgtccgcgccaaccccaagtccgatcacttcgccgtgaagcggttccaccacattgagttctggtgcggcgacgccaccaacacgtcgcggcggttctcgtggggcctcggcatgcctttggtggcgaaatcggatctctctactggaaactctgttcacgcttcttatcttgttcgctcggcgaatctcagtttcgtcttcaccgctccttactctccgtccacgaccacttcctctggttcagctgccatcccgtctttttcggcatcgggttttcactcttttgcggccaaacacggccttgctgttcgggctattgctcttgaagttgctgacgtggctgctgcgtttgaggccagtgttgcgcgtggggccaggccggcttcggctcctgttgaattggacgaccaggcgtggttggctgaggtggagttgtacggagatgtggtcttgaggtttgttagttttgggagggaggagggtttgtttttgcctggattcgaggcggtggaggggacggcgtcgtttccggatttggattatggaattagaagacttgatcatgcggtggggaatgttaccgagttggggcctgtggtggagtatattaaagggtttacggggtttcatgaatttgcggagtttacagcggaggatgtggggactttggagagtgggttgaattcggtggtgttggcgaataatgaggagatggttctgttgcccttgaatgagcctgtgtatgggaccaagaggaagagtcagatacagacttacttggagcacaatgaaggggctggagtgcagcatttggctttagtgagtgaggatatttttaggactttaagggagatgaggaagaggagttgccttggtggttttgagtttatgccttcgccaccgcctacgtattacaagaatttgaagaatagggtcggggatgtgttgagtgatgaacagatcaaggagtgtgaagatttggggattttggtggatagggatgatcagggtacattgcttcaaatctttaccaagcctgtaggtgacaggcctaccttattcatagagatcattcagagggtagggtgcatgctcaaggacgatgcagggcagatgtaccagaagggcgggtgcggaggatttgggaaggggaacttctcagagctgttcaagtccatcgaagaatatgaaaaaacacttgaagctaaacaaatcactggatctgctgctgcatga

SEQ ID NO: 16 - protein, 442 aa, Daucus carota
MGKKQSEAEILSSNSSNTSPATFKLVGFNNFVRANPKSDHFAVKRFHHIEFWCGDATNTSRRFSWGLGMPLVAKSDLSTGNSVHASYLVRSANLSFVFTAPYSPSTTTSSGSAAIPSFSASGFHSFAAKHGLAVRAIALEVADVAAAFEASVARGARPASAPVELDDQAWLAEVELYGDVVLRFVSFGREEGLFLPGFEAVEGTASFPDLDYGIRRLDHAVGNVTELGPVVEYIKGFTGFHEFAEFTAEDVGTLESGLNSVVLANNEEMVLLPLNEPVYGTKRKSQIQTYLEHNEGAGVQHLALVSEDIFRTLREMRKRSCLGGFEFMPSPPPTYYKNLKNRVGDVLSDEQIKECEDLGILVDRDDQGTLLQIFTKPVGDRPTLFIEIIQRVGCMLKDDAGQMYQKGGCGGFGKGNFSELFKSIEEYEKTLEAKQITGSAAA

SEQ ID NO: 17 - DNA, 1146 bp, Streptomyces avermitilis
  CDS (1)..(1146)
atgacgcagaccacacaccacactcccgacaccgcccggcaggccgaccccttcccggtgaagggaatggacgcggtcgtcttcgccgtaggcaacgccaagcaggccgcgcactactactccaccgccttcggcatgcagcttgtggcgtactccggaccggagaacggcagccgcgagaccgcttcgtacgtcctcaccaacggctcggcacgcttcgtcctcacctccgtcatcaagcccgccaccccctggggccacttcctcgccgaccatgtggccgagcacggcgacggcgtcgtcgacctcgccatcgaggtcccggacgcccgcgccgcccacgcgtacgcgatcgagcacggcgcccgctcggtcgccgagccgtacgagctgaaggacgagcacggcacggtcgtcctcgccgcgatcgccacctacggcaagacccgccacaccctcgtcgaccggaccggctacgacggcccctacctccccggctacgtggccgccgccccgatcgtcgaaccgcccgcccaccgcaccttccaggccatcgaccactgcgtcggcaacgtcgagctcggccggatgaacgaatgggtcggcttctacaacaaggtcatgggcttcacgaacatgaaggagttcgtgggcgacgacatcgcgaccgagtactcggcgctgatgtcgaaggtcgtggccgacggcacgctcaaggtcaagttcccgatcaacgagcccgccctcgccaagaagaagtcccagatcgacgagtacctggagttctacggcggcgcgggcgtccagcacatcgcgctgaacacgggtgacatcgtcgagacggtacgcacgatgcgcgccgccggcgtccagttcctggacacgcccgactcgtactacgacaccctcggggagtgggtgggcgacacccgcgtccccgtcgacaccctgcgcgagctgaagatcctcgcggaccgcgacgaggacggctatctgctccagatcttcaccaagccggtccaggaccgcccgacggtcttcttcgagatcatcgaacgccacggctcgatgggattcggcaagggcaacttcaaggccctgttcgaggcgatcgagcgggagcaggagaagcggggcaacctgtag

SEQ ID NO: 18 - protein, 381 aa, Streptomyces avermitilis
MTQTTHHTPDTARQADPFPVKGMDAVVFAVGNAKQAAHYYSTAFGMQLVAYSGPENGSRETASYVLTNGSARFVLTSVIKPATPWGHFLADHVAEHGDGVVDLAIEVPDARAAHAYAIEHGARSVAEPYELKDEHGTVVLAAIATYGKTRHTLVDRTGYDGPYLPGYVAAAPIVEPPAHRTFQAIDHCVGNVELGRMNEWVGFYNKVMGFTNMKEFVGDDIATEYSALMSKVVADGTLKVKFPINEPALAKKKSQIDEYLEFYGGAGVQHIALNTGDIVETVRTMRAAGVQFLDTPDSYYDTLGEWVGDTRVPVDTLRELKILADRDEDGYLLQIFTKPVQDRPTVFFEIIERHGSMGFGKGNFKALFEAIEREQEKRGNL

SEQ ID NO: 19 - DNA, 45 bp, Artificial
  Synthetic primer sequence
ccatggctcatcaccatcaccatcaccaaaacgccgccgtttcag

SEQ ID NO: 20 - DNA, 27 bp, artificial
  Synthetic primer sequence
tctagatcatcccactaactgtttggc

SEQ ID NO: 21 - DNA, 51 bp, artificial
  Synthetic primer sequence
ccatggctcatcaccatcaccatcacgcagatctatacgaaaacccaatgg

SEQ ID NO: 22 - DNA, 29 bp, artificial
  Synthetic primer sequence
tctagattaatcggcggtcaatacaccac

SEQ ID NO: 23 - DNA, 33 bp, artificial
  Synthetic primer sequence
  misc_feature (16)..(18): n is a, c, g, or t
ggtggttttggcaaannnaatttctctgagctc

SEQ ID NO: 24 - DNA, 33 bp, artificial
  Synthetic primer sequence
  misc_feature (16)..(18): n is a, c, g, or t
gagctcagagaaattnnntttgccaaaaccacc

SEQ ID NO: 25 - DNA, 33 bp, artificial
  Synthetic primer sequence
  misc_feature (16)..(18): n is a, c, g, or t
cagcgccttgaagttnnnctcgccaaacccatc

SEQ ID NO: 26 - DNA, 33 bp, artificial
  Synthetic primer sequence
  misc_feature (16)..(18): n is a, c, g, or t
gatgggtttggcgagnnnaacttcaaggcgctg

SEQ ID NO: 27 - DNA, 22 bp, artificial
  Synthetic primer sequence
gatcttctcggaaaccctgatg

SEQ ID NO: 28 - DNA, 22 bp, artificial
  Synthetic primer sequence
gggattcttgtagacagagatg

SEQ ID NO: 29 - DNA, 20 bp, artificial
  Synthetic primer sequence
cccactaactgtttggcttc

SEQ ID NO: 30 - DNA, 20 bp, artificial
  Synthetic primer sequence
ggcggtcaatacaccacgac

SEQ ID NO: 31 - DNA, 23 bp, artificial
  Synthetic primer sequence
gactcgaacagcgccttgaagtt

SEQ ID NO: 32 - DNA, 18 bp, artificial
  Synthetic primer sequence
ggatgtggtggttttggc